SEQUENCE LISTING (24 sequences)

SEQ ID NO: 1 - protein, 16 aa, synthetic construct
  REGION 1..16: note = Description of Artificial Sequence: Synthetic Peptide
RADARADARADARADA

SEQ ID NO: 2 - protein, 13 aa, synthetic construct
  REGION 1..13: note = Description of Artificial Sequence: Synthetic Peptide
IEIKIEIKIEIKI

SEQ ID NO: 3 - protein, 12 aa, synthetic construct
  REGION 1..12: note = Description of Artificial Sequence: Synthetic Peptide
QLELQLELQLEL

SEQ ID NO: 4 - protein, 12 aa, synthetic construct
  REGION 1..12: note = Description of Artificial Sequence: Synthetic Peptide
KLDLKLDLKLDL

SEQ ID NO: 5 - protein, 16 aa, synthetic construct
  REGION 1..16: note = Description of Artificial Sequence: Synthetic Peptide
RATARAEARATARAEA

SEQ ID NO: 6 - protein, 16 aa, synthetic construct
  REGION 1..16: note = Description of Artificial Sequence: Synthetic Peptide
ADARADARADARADAR

SEQ ID NO: 7 - protein, 16 aa, synthetic construct
  REGION 1..16: note = Description of Artificial Sequence: Synthetic Peptide
RAEARAEARAEARAEA

SEQ ID NO: 8 - protein, 16 aa, synthetic construct
  REGION 1..16: note = Description of Artificial Sequence: Synthetic Peptide
EARAEARAEARAEARA

SEQ ID NO: 9 - protein, 16 aa, synthetic construct
  REGION 1..16: note = Description of Artificial Sequence: Synthetic Peptide
RVDVRVDVRVDVRVDV

SEQ ID NO: 10 - protein, 16 aa, synthetic construct
  REGION 1..16: note = Description of Artificial Sequence: Synthetic Peptide
RLDLRLDLRLDLRLDL

SEQ ID NO: 11 - protein, 16 aa, synthetic construct
  REGION 1..16: note = Description of Artificial Sequence: Synthetic Peptide
RIDIRIDIRIDIRIDI

SEQ ID NO: 12 - protein, 16 aa, synthetic construct
  REGION 1..16: note = Description of Artificial Sequence: Synthetic Peptide
RFDFRFDFRFDFRFDF

SEQ ID NO: 13 - protein, 16 aa, synthetic construct
  REGION 1..16: note = Description of Artificial Sequence: Synthetic Peptide
AEARAEARAEARAEAR

SEQ ID NO: 14 - protein, 16 aa, synthetic construct
  REGION 1..16: note = Description of Artificial Sequence: Synthetic Peptide
KADAKADAKADAKADA

SEQ ID NO: 15 - protein, 16 aa, synthetic construct
  REGION 1..16: note = Description of Artificial Sequence: Synthetic Peptide
KIDIKIDIKIDIKIDI

SEQ ID NO: 16 - protein, 16 aa, synthetic construct
  REGION 1..16: note = Description of Artificial Sequence: Synthetic Peptide
KIEIKIEIKIEIKIEI

SEQ ID NO: 17 - protein, 13 aa, synthetic construct
  REGION 1..13: note = Description of Artificial Sequence: Synthetic Peptide
IDIKIDIKIDIKI

SEQ ID NO: 18 - protein, 13 aa, synthetic construct
  REGION 1..13: note = Description of Artificial Sequence: Synthetic Peptide
IEIRIEIRIEIRI

SEQ ID NO: 19 - protein, 13 aa, synthetic construct
  REGION 1..13: note = Description of Artificial Sequence: Synthetic Peptide
LELKLELKLELKL

SEQ ID NO: 20 - protein, 13 aa, synthetic construct
  REGION 1..13: note = Description of Artificial Sequence: Synthetic Peptide
FEFKFEFKFEFKF

SEQ ID NO: 21 - protein, 12 aa, synthetic construct
  REGION 1..12: note = Description of Artificial Sequence: Synthetic Peptide
KLELKLELKLEL

SEQ ID NO: 22 - protein, 13 aa, synthetic construct
  REGION 1..13: note = Description of Artificial Sequence: Synthetic Peptide
FEFRFEFRFEFRF

SEQ ID NO: 23 - protein, 13 aa, synthetic construct
  REGION 1..13: note = Description of Artificial Sequence: Synthetic Peptide
YEYKYEYKYEYKY

SEQ ID NO: 24 - protein, 13 aa, synthetic construct
  REGION 1..13: note = Description of Artificial Sequence: Synthetic Peptide
WEWKWEWKWEWKW

(19) United States
(12) Patent Application Publication
(10) Pub. No.: US 2023/0381185 A1
(43) Pub. Date: Nov. 30, 2023

(54) NOVEL FORMULATIONS FOR ORAL ADMINISTRATION OF THERAPEUTIC AGENTS TO THE GASTROINTESTINAL TRACT

(71) Applicants: 3-D Matrix, Ltd., Cambridge, MA (US); UNIVERSITE CLAUDE BERNARD LYON 1, Villeurbanne (FR); Hôpital Lyon Sud, Pierre-Bénite (FR)

(72) Inventors: Giovanna Lollo, Lyon (FR); Marie Reveiller Buffier, Fountaines Saint Martin (FR); Sharanya Sankar, Rillieux La Pape (FR); David Kryza, Saint Didier Au Mont D'Or (FR); Annalisa Rosso, Villeurbanne (FR); Vanentina Andretto, Lyon (FR); Stephane Nancey, Charley (FR); Gilles Boschetti, Brindas (FR)

(73) Assignees: 3-D Matrix, Ltd., Tokyo (JP); UNIVERSITE CLAUDE BERNARD LYON 1, Villeurbanne (FR); Hôpital Lyon Sud, Pierre-Bénite (FR)

(21) Appl. No.: 18/317,002

(22) Filed: May 12, 2023

Related U.S. Application Data
(60) Provisional application No. 63/342,081, filed on May 14, 2022.
(60) Provisional application No. 63/412,464, filed on Oct. 2, 2022.

(51) Int. Cl. A61K 31/519; A61K 9/107; A61K 9/51; A61K 9/00; A61P 1/06

(52) U.S. Cl. CPC A61K 31/519; A61K 9/1075; A61K 9/51; A61K 9/0053; A61P 1/06

(57) ABSTRACT

Described herein is a novel nanocomposite for oral administration for treatment of gastrointestinal disease, e.g., IBD, or injury. The nanocomposite contains nanoemulsion particles (NEs), loaded with an active therapeutic agent, that are then combined with a second component containing a self-assembling peptide(s) (SAP) or peptidomimetic(s) (optionally, the second component is in a powder form). Upon administration, when hydrated at slightly acidic or near-neutral pH, the system self-assembles into a hydrogel matrix enveloping NEs, which then adheres to, and more efficiently delivers, the active agent to the intestinal tract of a treated subject. The nanocomposite can be administered in a capsule dosage form that forms a depot in the stomach or post-stomach.

### PRIORITY

This Application claims priority to US provisional Application Nos. 63/342,081, filed May 14, 2022, and 63/412,464, filed Oct. 2, 2022, the entire contents of each of which are hereby incorporated by reference in their entirety.

### SEQUENCE LISTING

The instant application contains an XML Sequence Listing which has been submitted electronically and is hereby incorporated by reference in its entirety. The Sequence Listing, created on Apr. 10, 2023, is named 3DM-21-06-ORAL-US_SL.xml and is 31,761 bytes in size.

### FIELD OF THE INVENTION

The present disclosure relates to methods of making and uses of drug formulations for treatment of diseases or injuries of the gastrointestinal tract.

### BACKGROUND

Inflammatory Bowl Diseases (IBDs) often require prolonged treatments to promote mucosal healing, and to induce and maintain long-term clinical remission. The therapeutic options include corticosteroids, 5-aminosalicylates (5-ASA), immunomodulators, immunosuppressants, and/or therapeutic agents. While numerous treatment options are available, many can provoke drug class-related complications, with patients potentially becoming intolerant of treatment over time (Lefevre, P. L. C. & Casteele, N. Vande. Clinical pharmacology of janus kinase inhibitors in inflammatory bowel disease. Journal of Crohn's and Colitis vol. 14 S725-S736 (2020)). With an improved understanding of the involvement of inflammatory cytokines in the pathogenesis of inflammatory bowel disease, janus kinase (JAK) inhibitors have emerged as an effective oral treatment option for ulcerative colitis. By inhibiting the cytokine-activated JAK component of the JAK-signal transducers and activators of transcription (STATs) pathway, JAK inhibitors interfere with the signaling of a variety of cytokines involved in the aberrant immune response contributing to the development of IBD. (Zundler, S. & Neurath, M. F. Integrating immunologic signaling networks: The JAK/STAT pathway in colitis and colitis-associated cancer. Vaccines Vol. 4 (2016). Tofacitinib (Xeljanz®) is a first-in-class, small molecule JAK inhibitor approved for the treatment of moderate to severe ulcerative colitis in the EU and USA.

Oral administration of therapeutics is the preferred route, improving safety, convenience, and patient compliance. Moreover, the oral route facilitates the local drug delivery and systems accumulation in the gastrointestinal tract (GIT). However, the oral bioavailability of many drugs is limited by various biological barriers. To enhance therapeutic activity, especially for localized application, attempts to develop various hybrid systems resulting in a prolonged drug residence time in the gastrointestinal tract, thereby leading to increased drug delivery, has been described, for example, in Sharma, S. & Sinha, V. R. Current pharmaceutical strategies for efficient site specific delivery in inflamed distal intestinal mucosa. Journal of Controlled Release vol. 272 97-106 (2018) and include, for example, polymethacrylate delivery systems, carbohydrate-based delivery systems, in particular and vesicular systems. In a work previously reported by Rosso et al., Control. Release (2021) 333: 579-592), nanocomposite sponges based on a naturally occurring polysaccharide, namely, chitosan, were evaluated for enhancing intestinal residence time following oral administration.

More recently, drug-carrying nanoparticles have been increasingly combined with hydrogels. These hydrogels are commonly made of polymers such as above-mentioned chitosan, alginate, dextran, carrageenan, polycaprolactone (PCL), and hydroxypropyl methylcellulose (HPMC) to form hybrid systems for controlled or enhanced drug delivery to the gut (Andretto, V., Rosso, A., Briancon, S. & Lollo, G. Nanocomposite systems for precise oral delivery of drugs and biologics. Drug Deliv. Transl. Res. 11, 445-470 (2021)).

Hydrogels represent in the macro- and microscale the most used type of nanocomposite in oral delivery. However, soft ingestible hydrogels can face practical concerns, notably pH-dependent behavior, mechanical weakness and excessive swelling speed, which shorten their GI residence time and have limited storage stability. The use of nanocomposite as multicompartimental capsules, tablets, aerogels, sponges, and films have been described. The main requirement of such solid systems is that following reconstitution in GI media, both the nanoparticles and the polymeric matrixes must recover the initial properties and recognize specific target sites exerting their activity. This implies a careful selection of the constituents of the nanocomposites focusing on their FDA approval for oral route, physico-chemical properties, structural characteristics (crystallinity, fluidity) and interactions between them. Another important aspect is the understanding and prediction of nanoparticles release from nanocomposites. The main strategies described are based on pH triggered matrix degradation or dissolution, chemically or enzymatically driven matrix erosion, pH or temperature dependent swelling of the polymeric network, and the nanoparticle/drug diffusion or desorption. For a better understanding of the release dynamics, the effective diffusion coefficients inside different polymeric matrix should be estimated by modelling the diffusion process.

Therefore, there remains a need to develop additional drug delivery systems for improved treatment of gastrointestinal diseases or injuries.

### SUMMARY OF THE INVENTION

A hybrid system composed of mucopenetrating lipid nanoparticles (NE) embedded in the self-assembling peptide hydrogel was developed. Self-assembling peptides are described, for example, in U.S. Pat. No. 9,724,448, herein incorporated by reference. The self-assembling peptide, RADARADARADARADA (SEQ ID NO:1) also known as RADA16 (“2.5% v/w RADA16-containing product, called PURASTAT (also referred herein as “PS” or “PM”, used interchangeably), available from 3-D Matrix, Ltd.; www.3dmatrix.com) was chosen as model self-assembling peptide and used as fractional concentrations as indicated. Tofacitinib (TFC) was chosen as model drug for the proof of concept, however, any other small hydrophobic or any suitable other drugs could be used. In another embodiment, of the current invention the drug is budesonide, a cortisone-like hydrophobic drug. Other small-molecule drugs, for example, listed in the publication of Fitzpatrick et al., such as S1P receptor modulators, other JAK inhibitors, CCR9 antagonists, alpha-4 integrin antagonists, immunomodulators, can ideally be encapsulated in the nanosystem for the creation of different nanocomposites (Fitzpatrick, L. R., and T. Woldemariam. “Small-molecule drugs for the treatment of inflammatory bowel disease.” (2017): 495-510.) The hybrid system can transport and release selected mucopenetrating drug-loaded nanosystems to the intestinal wall, thereby maximizing the local effective drug dosage by controlling the permeability and enhancing the biological stability of the drug as well as providing rapid induction of hemostasis by the hydrogel.

The use of the hybrid system comprised of PS hydrogel has at least two separate advantages: a prolonged residence time in the GIT leading to an increased drug delivery, and the epithelial and tissue regeneration enhanced by the properties of the hydrogel, with properties resembling the ones of the extracellular matrix.

Additionally, certain disease factors were improved by administration of PS hydrogel/NE mixture alone, in the absence of any active ingredients.

### BRIEF DESCRIPTION OF THE FIGURES

FIG. 1 is a schematic of a possible mechanism of the invention in treating inflamed/damaged intestinal tissue.

FIGS. 2A-C illustrate data from rheological characterization of hydrogels with and without embedded NEs. FIG. 2A shows the amplitude dependence of moduli. The mean (n=3) and standard deviation bars are shown. FIG. 2B shows average mesh size and FIG. 2C shows crosslinking density of the hydrogels at different concentrations before and after embedding with NEs. The mean (n=3) and standard deviation bars are shown.

FIG. 3 provides data from Circular Dichroism (CD) studies of PS and PS loaded with NEs under different conditions.

FIG. 4: shows cumulative release of Curcumin from PS loaded with NE-Curcumin particles under various conditions in vitro.

FIG. 5 shows Phase I results for DSS induction of inflammation and tissue damage in mice. The results showed that an administration of 3% v/v DSS in water over a period of 7 days led to a too severe colitis induction, while administration for 4 days led to a slow and complete recovery of the animals. In fact, in these studies it is important not to induce an extreme condition that will be too difficult to treat. For this reason, an incubation time with DSS over 4 days has been chosen for the next studies, followed by treatment until day 10 that showed a good recovery in terms of macroscopic and histologic evaluations.

FIG. 6 shows fluorescence images of excised GIT of healthy and DSS treated mice sacrificed after oral gavage with DiD-NEs in solution or DiD-NEs loaded in PS. Ex vivo images of the GIT of the healthy (upper line) and IBD (bottom line) mice treated with NEs alone (panel on the left) or PS-NEs hybrid system (panel on the right).

FIGS. 7A and 7B show quantitation of location of fluorescence intensity in GIT from mice treated with (FIG. 7A) NE-DiD or (FIG. 7B) PS loaded with NE-DiD as shown for example in FIG. 6. FIGS. 7A and 7B show semi-quantitative analysis of the fluorescence from a picture taken immediately after animal dissection and analyzed using Wasabi software. FIG. 7A shows results for NE administration to healthy and IBD mice; FIG. 7B shows results for PS-NE composite administration to healthy and IBD mice.

FIGS. 8A and 8B: The qualitative fluorescence obtained by LSFM (light sheet fluorescence microscopy) after tissue clarification was done only on two IBD mice that received NE-DiD or PS+NE-DiD and were sacrificed at 3 hours. The upper section of the colon of IBD mice which received the NE in solution (shown in FIG. 8A) or the PS-NE (shown in FIG. 8B) by oral gavage. The tissue was collected and fixed in PFA 4% v/v for 24 h, before being transferred in PBS, pH 7.4 for the qualitative visualization of the preferential accumulation of the system. X-clarity tissue clearing system was used to clarify the tissues and light sheet fluorescence microscopy was used to observe the fluorescence of the particles in the colon.

FIGS. 9A and 9B show results from Phase II in vivo studies of mice (N=10 per group) administered 3% DSS in drinking water for 4 days and then treated with various formulations compared to healthy controls until sacrifice at 10 days. FIG. 9A shows percent weight loss, and FIG. 9B shows the DAI score.

FIG. 10: DAI score for IBD mice (treated with DSS) showing a statistically significant improvement at days 7 and 8 for mice subsequently administered PS loaded with NEs with Tofacitinib (TFC) relative to the other groups.

FIGS. 11A and 11B: In FIG. 11A, the experimentally observed ratio between the weight and the length of the animals' colon sacrificed at day 10 is shown. As displayed from the ex vivo image FIG. 11B, the colon of inflamed animals (G2) is shorter in terms of length when compared with the negative control group (G1), and it shows presence of blood and faeces not completely formed. It was noted also that the inflamed colons were heavier and less elastic than the healthy ones, probably due to the presence of oedematous changes and granulomatosis derived from the inflamedstate (Kim, H. Y. et al. Rumex japonicus Houtt. alleviates dextran sulfate sodium-induced colitis by protecting tight junctions in mice. Integr. Med. Res. 9, 100398 (2020)). However, treatment with TFC alone or in the nanocomposite at the same concentration (10 mg/kg) significantly prevented it (p<0.02). Gross evaluation of colons excised from mice in the various experimental groups (N=10). FIG. 11A shows weight of the colon (g) divided by length measured in cm is shown, for example, in FIG. 11B.

FIG. 12: Myeloperoxidase (MPO) activity, as an indicator of neutrophil infiltration in the colonic mucosa.

FIG. 13: HPS staining. Group 1 (G1): Healthy mice (n=5), negative control. The scale bar is 100 μm.

FIG. 14: HPS staining. Group 2 (G2): DSS-induced colitis mice (n=15), positive control. The scale bar is 100 μm.

FIG. 15: HPS staining. Group 3 (G3): DSS-induced colitis mice (n=10), oral gavage with TFC. The scale bar is 100 μm.

FIG. 16: HPS staining. Group 4 (G4): DSS-induced colitis mice (n=10), oral gavage with TFC-loaded NE (TFC_NE). The scale bar is 100 μm.

FIG. 17: HPS staining. Group 5 (G5): DSS-induced colitis mice (n=10), oral gavage with TFC-loaded NE embedded in PS (PM_NE_TFC). The scale bar is 100 μm.

FIG. 18: HPS staining. Group 6 (G6): DSS-induced colitis mice (n=10), oral gavage with empty PS. The scale bar is 100 μm.

FIG. 19A: Colon weight/colon length ratio average per group of mice (n=5).

FIG. 19B: Myeloperoxidase (MPO) activity, as an indicator of neutrophil infiltration in the colonic mucosa.

FIG. 19C: CD45⁺ cells/mm² quantified from the histology sections.

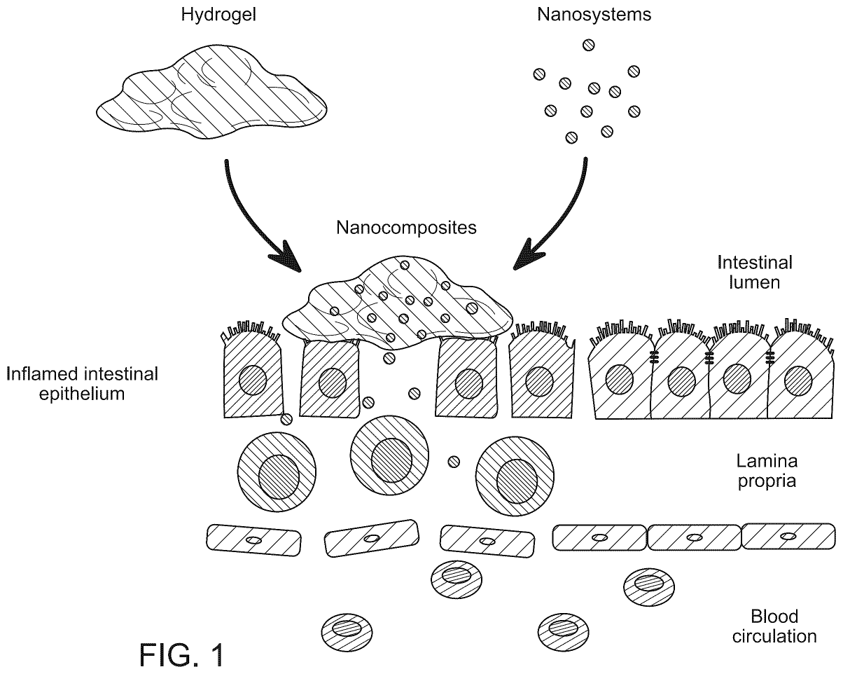

### DETAILED DESCRIPTION OF THE INVENTION

Following the development and characterization of the system described below, a mouse model generally accepted for in vivo studies of therapy directed at pathologies involving intestinal inflammation was utilized as proof of efficacy of the nanocomposite system for the desired indication-in the area of chronic inflammation of the gastrointestinal tract (GIT), especially related to the family of illnesses under the umbrella term of inflammatory bowel diseases (IBDs). The use of a hybrid system comprising PURASTAT (PM; PS, RADA16-(SEQ ID NO:1)) hydrogel, and more generally other suitable self-assembling peptides (SAPs) as described, has a number of additional advantages: a prolonged residence time in the GIT, an increment of the local drug delivery by offering a double encapsulation strategy, and epithelium and tissue regeneration promoted by of the hydrogel itself. In fact, treatment with PURASTAT has also been shown to result in rapid hemostasis for oozing wounds and reduction of delayed bleeding not dependent on the natural clotting process in other indications (references) such as surgical interventions even when the patient's natural hemostasis mechanism is impaired or dysfunctional.

An additional benefit of this nanocomposite system is that it can be dried and then reconstituted in vivo or under in vivo-like conditions without substantial alteration of its properties. The dried nanocomposite offers the advantage of a long-duration storage, but, perhaps more importantly, the dried nanocomposite powder can be encapsulated in various types of capsules known in the art for a controlled and localized delivery in precise part of the GIT with systems differentiated in the basis of GIT transit times, pH changes, bacterial enzymes, mucoadhesion, nanotechnologies and disease-associated triggers. One such capsule, designed for release of hydrated RADA16 hydrogel doped with a macromolecular dye, fluorescein isothiocyanate—dextran 4 kDa (FD4), mostly in the large intestine, has been described by Eleftheriadis et al. (Pharm. Development Tech. 25(4): 1-23 (2020)). That reference discloses a pH sensitive hydroxypropyl methylcellulose-phthalate (HPMCP)-based 3D-printed capsule. Other encapsulation materials suited to the present invention include poly(meth) acrylate polymers and members of the Eudragit family (Chu, J. N. and Traverso, G., Foundations of gastrointestinal-based drug delivery and future developments, Nature Reviews: Gastroenterology & Hepatology (2021) doi: 10.1038/s41575-021-00539-w). The present invention is based, at least, on the following findings/parameters:
1. A nanocomposite based on the physical mixture of a preformed nanosystem (namely nanoemulsion, NE) loaded with a drug intended for the treatment of IBD, and a preformed self-assembling-peptide (BASED)-based hydrogel (PURASTAT) was created at different concentrations of SAP.
2. The creation of nanocomposites characterized by two different concentrations, led to the development of systems with different structural properties, leading to differences in the drug-release behaviours.
3. The presence of the nanosystem did not alter the secondary structure of the peptide, of key importance for the hydrogel formation and maintenance of its structure.
4. The use of nanocomposite to test the therapeutic efficacy of the drug on an IBD-mouse model brought results based on different technique, detailed herein.
5. Certain disease factors were improved by administration of PS hydrogel/NE mixture alone, even in the absence of any active ingredients.
   Disease activity index (DAI) score, used to evaluate the DSS-induced colitis. Daily, animals were observed for weight loss, stool consistency and presence of gross blood faeces and anus. For each parameter a score from 0 to 4 was attributed and afterwards divided by three, resulting in the total DAI score ranging from 0 (unaffected) to 4 (severe colitis) (Gonçalves, F. da C., Schneider, N., H. M.-A. S. & 2013, U. Characterization of Acute Murine Dextran Sodium Sulfate (DSS) Colitis: Severity of Inflammation is Dependent on the DSS Molecular Weight and Concentration. Acta Sci. Vet. 41, 1-9 (2013)). Colon length and weight, which is often reduced in cases of inflammation (Biton, I. E. et al. Assessing Mucosal Inflammation in a DSS-Induced Colitis Mouse Model by MR Colonography. Tomogr. (Ann Arbor, Mich.) 4, 4-13 (2018)). The activity of the enzyme myeloperoxidase (MPO), which is a direct indicator of neutrophils infiltration into the colonic mucosa, and thus an important marker of inflammation (Hansberry, D. R., Shah, K., Agarwal, P. & Agarwal, N. Fecal Myeloperoxidase as a Biomarker for Inflammatory Bowel Disease. Cureus 9, (2017)). Histomorphologic evaluation by HPS stain (hematoxylin phloxine saffron stain), similar to H&E. HPS can differentiate between the most common connective tissue (collagen) and muscle and cytoplasm by staining the former yellow and the latter two pink, unlike an H&E stain, which stains all three pink. Immunohistochemistry for the detection of CD45 positive cells.

### Formulation and Characterization of Nanoemulsion (NE) Particles

The composition, formulation, and characterization of the nanoemulsion particles as component of the present invention have been mostly previously described by Rosso et al. (Development and structural characterization of a novel nanoemulsion for oral drug delivery, Colloids and Surfaces A (2020) 593: 124614). As described in that reference, NE were prepared by emulsion phase inversion (EPI) technique coupled with high stirring energy input. Briefly, NE were composed of medium chain triglycerides (MCT) (Miglyol®812) oil purchased from CREMER OLEO GmbH & Co. KG (Hamburg, Germany) core stabilized by a surfactant shell made of a mixture of hydrophilic and hydrophobic surfactants, namely polyoxyethylene (40) stearate (Myrj®52), identified herein as S1, and oleoyl polyoxyl-6 glycerides (Labrafil®1944CS)-identified as S2, respectively. To prepare the oil phase, MCT (0.35 g) and surfactants (1 g) were mixed and magnetically stirred (750 rpm) using a thermostated bath at 80° C. The aqueous phase (PBS 5 mM, 3.65 mL), heated up to 80° C. as well, was added into the organic melt phase. Stirring was then performed by two cycles of 10 min using a rotor-stator disperser (T25 digital Ultra-Turrax® equipped with a S25N-10G shaft, IKA®-Werke GmbH & Co. KG, Staufen, Germany) rotating at 11000 rpm at 80° C.

As described therein, medium chain triglycerides, MCT (Miglyol®812) purchased from CREMER OLEO GmbH & Co. KG (Hamburg, Germany) were used as oil forming the NE core. Polyoxyethylene(40) stearate (Myrj®52), identified herein as S1, from Sigma-Aldrich (St Quentin-Fallavier, France) and oleoyl polyoxyl-6 glycerides (Labrafil®1944CS), identified herein as S2, from Gattefosse (Saint-Priest, France) were used as nonionic surfactants which comprise the outer shell of the NEs. The aqueous phase used to prepare emulsions was sodium phosphate buffer solution (5 mM; pH 7.4). Tacrolimus, a BCS Class II immunomodulatory agent used in the treatment of various diseases, chosen as hydrophobic model drug to be encapsulated into NE in these studies, was purchased from LC Laboratories (Woburn, MA, USA).

The chosen materials and their ratio were the results of a series of experiment described by Rosso, et al. (2020) To investigate the NE region, three ternary phase diagrams are designed using 23 formulations for each diagram. The ternary mixtures are composed of oil, water and three different surfactant mixtures (S1+S2) called Smix. Smix was characterized by the Surfactant Mass Ratio (SMR) of S1 to S2.

SMR=mass of S1/mass of S2 Eq (1)

The NE area was identified by varying the Smix/Oil/Water amount at fixed SMR of 1, 2.5, and 5. Another parameter of the formulation was the Surfactant-to-Oil ratio (SOR) defined as

SOR=mass of Smix/mass of MCT Eq (2)

In the reported studies, the different NE comprised as just described were exhaustively characterized according to electrical conductivity, shear viscosity, X-ray powder diffraction (XRPD) analysis, size distribution, surface electrical potential, morphology and other features by methods known in the art. Based on analysis of the results of these experiments, further experiments and analyses were undertaken to select NE compositions for further use. Optimized NE had mean droplet size of around 104±3 nm, a low polydispersity index (PDI) of (0.2) and a neutral/slightly negative zeta potential (−9±1 mV). This neutral surface charge derived from the PEGylated surfactant (S1) shell hinders interactions with intestinal contents and mucus layers, thus enhancing NE diffusion to the epithelium and translocation through the mucosa (Hua, et al., Advances in oral nano-delivery systems for colon targeted drug delivery in inflammatory bowel disease: selective targeting to diseased versus healthy tissue, Nanomed. Nanotechnol., Biol. Med. 11 (2015) 1117-1132; doi.org/10.1016/j.nano.2015.02.018).

As an example of further use of optimized NE reported by Rosso et al. (2020), tacrolimus, an immunomodulatory agent used in the treatment of various diseases (Zhang et al., Multifunctional Poly(methyl vinyl ether-comaleic anhydride)-graft-hydroxypropyl-β-cyclodextrin amphiphilic copolymer as an oral high-performance delivery carrier of tacrolimus, Mol. Pharm. 12 (2015) 2337-2351; doi.org/10.1021/acs.molpharmaceut.5b00010), was encapsulated into the NE as a hydrophobic model drug. The tacrolimus-loaded NE were observed to be relatively stable in storage at 20 and 37° C. for 28 days and demonstrated delayed release of the drug relative to the drug dissolved in solution in vitro in a simulated GI environment.

Tofacitinib-loaded NEs as used in the in vivo efficacy studies disclosed below were prepared by dissolving the drug in the organic phase, in order to reach a final concentration of 4 mg/mL. DiD (fluorescent label)-loaded NEs were prepared by adding that carbocyanine derivative fluorescent dye in the organic phase to obtain a final concentration of 0.057 mg/mL for the biodistribution in vivo studies disclosed in this patent application. Table 1 shows the characteristics of the NEs prepared by the method described by Rosso et al. (2020).

TABLE 1

| Characteristic | |
|---|---|
| Particles type | Nanoemulsions (NE) |
| Preparation method | EPI + high stirring energy |
| Oil phase | 25% Oil + 75% Surfactants |
| Additional solvents | — |
| Water phase | PBS 5 mM |
| Size (nm) | 104 ± 3 |
| PDI | 0.2 |
| Surface charge (mV) | −9 ± 1 |

The size distribution and surface potential of the NE droplets were determined using Malvern Zetasizer® Nano ZS instrument (Malvern Instruments S.A., Worcestershire, UK). The particle sizes were measured by Dynamic Light Scattering (DLS) at 25° C. at a scattering angle of 173°. The ξ-potential was calculated from the mean electrophoretic mobility measured for samples diluted in milliQ water. The stability of blank and TFC-NEs in colloidal suspension was followed during 6 months upon storage at 4° C. At scheduled time points, particle size, polydispersity index (PDI), and ξ-potential were measured.

### General Rheological and Structural Characteristics of PURASTAT Loaded with Nanoemulsion Particles (NEs) to Form Nanocomposites

Formulating the nanocomposite, the nanoemulsion (NE) was efficiently loaded in PURASTAT (SEQ ID NO:1) at different SAP concentrations, 0.5% and 2.0% w/v. (Of note, when referencing the amount of the SAP or peptidomimetics (collectively referred to as “peptoproducts”) in solution (sometimes loosely referred to as “hydrogel” due the nature of the final product, while implying “solution”), the “v/w” r refers to the weight of the substantially pure (relative to its full-length “peptoproduct” (e.g., at least 65%, at least 70%, preferably, at least 75%, at least 80%, at least 85%, most preferably, at least 90%, at least 95%, or more pure) in solution prior to hydrogel formation.

Tofacitinib (TFC)-loaded NEs were prepared by dissolving the drug in the organic phase, in order to reach a final concentration of 4 mg/mL desired for in vivo studies with the nanocomposite. DiD-loaded NEs were prepared by adding this carbocyanine derivative fluorescent dye in the organic phase to obtain a final concentration of 0.5 and 0.057 mg/mL for the in vitro assays and the biodistribution in vivo studies, respectively.

IBD often requires prolonged treatment to promote mucosal healing and to induce and maintain long-term clinical remission. The treatment involves the use of corticosteroids, 5-aminosalicylates (5-ASA), immunomodulators, immunosuppressants and/or targeted biological agents, such as monoclonal antibodies. While numerous treatment options are available, many can provoke drug class-related complications, with patients potentially becoming intolerant of treatment over time [Pavine L C Lefevre, Niels Vande Casteele, Clinical Pharmacology of Janus Kinase Inhibitors in Inflammatory Bowel Disease, Journal of Crohn's and Colitis, Volume 14, Issue Supplement_2, July 2020, Pages S725-S736, https://doi.org/10.1093/ecco-jcc/jjaa014]. With an improved understanding of the involvement of inflammatory cytokines in the pathogenesis of inflammatory bowel disease, janus kinase (JAK) inhibitors have emerged as an effective oral treatment option for ulcerative colitis. By inhibiting the cytokine-activated JAK component of the JAK-signal transducers and activators of transcription (STATs) pathway, JAK inhibitors modulate the cytokines mediated aberrant immune response that contributes to the development of IBD [Zundler, S.; Neurath, M. F. Integrating Immunologic Signaling Networks: The JAK/STAT Pathway in Colitis and Colitis-Associated Cancer. Vaccines 2016, 4, 5.; see also doi.org/10.3390/vaccines4010005]. Tofacitinib (Xeljanz®) is a first-in-class, small molecule JAK inhibitor approved for the treatment of moderate to severe ulcerative colitis in the EU and USA. For these reasons, Tofacitinib (TFC) was chosen as model drug for the proof of concept of our project. In another embodiment of the current invention the drug is budesonide, a cortisone-like hydrophobic drug. Other small-molecule drugs for example listed in the publication of Fitzpatrick et al., such as S1P receptor modulators, other JAK inhibitors, CCR9 antagonists, alpha-4 integrin antagonists, immunomodulators, can ideally be encapsulated in the nanosystem for the creation of different nanocomposites (Fitzpatrick, L. R., and T. Woldemariam. “Small-molecule drugs for the treatment of inflammatory bowel disease.” (2017): 495-510.)

Tofacitinib was loaded in NEs the needed concentration for the therapeutic activity in mice (10 mg/kg). The optimisation of the formulation also took into account the stability of the system in terms of encapsulation efficiency, size, and surface charge, all important parameters for a stable and controlled drug release. The optimized formulation reached the drug loading (DL %) of 2.2%

The size distribution and surface potential of the NE droplets were determined using Malvern Zetasizer® Nano ZS instrument (Malvern Instruments S.A., Worcestershire, UK). The particle sizes were measured by Dynamic Light Scattering (DLS) at 25° C. at a scattering angle of 173°. The ξ-potential was calculated from the mean electrophoretic mobility measured for samples diluted in milliQ water. The stability of blank and TFC-NEs in colloidal suspension was followed during 6 months upon storage at 4° C. At scheduled time points, particle size, polydispersity index (PDI), and ξ-potential were measured.

To quantify the TFC loaded in the nanosystem, the NE was dissolved in MeOH to break the particles' structure and analysed by RP-HPLC. The liquid chromatography system consisted of UHPLC Aquity Arc with a diode array detector (PDA), binary pump and septum injection valve with fixed 10 μL loop. The analyte was monitored at 254 nm. Chromatographic analyses were carried out on a Kinetex C18 column 150 mm×4.6 mm in size and a particle size of 5 μm (Phenomenex, Torrance, CA, USA). In order to elute the compound, a mixture of methanol and water in a ratio of 50:50 v/v was used and the column temperature was set at 30° C. [VK, Dhiman V, Giri K K, Sharma K, Zainuddin M, Mullangi R. Development and validation of a RP-HPLC method for the quantitation of tofacitinib in rat plasma and its application to a pharmacokinetic study. Biomed Chromatogr. 2015 September; 29(9):1325-9. doi:10.1002/bmc.3426. Epub 2015 Jan. 26. PSID: 25622797.]. The HPLC calibration curve was linear (R²=0.99) in the concentration range of 20-100 μg/mL. The method was validated according to ICH Q2(R1) guidelines. Detection and quantification limits (LOD and LOQ) were 6.17 μg/mL and 18.69 μg/mL, respectively. The diluted samples were filtered using a 0.22 μm Nylon filter (Whatman GmbH, Dassel, Germany) before injection in the HPLC system. The drug concentration was followed for 6 months.

Nanocomposites have been created by mixing two concentrations of PS with a pre-defined amount of NE to observe different behaviors in terms of particles release, being the concentration of NE constant (5% w/v), to obtain final PS concentration of 0.5 and 2% w/v. The mixture of PS and NE, when necessary diluted with a saline solution at pH 1.8, was vortexed for 5 minutes to obtain a homogenous final formulation. The two final nanocomposites will be defined as NE_PM0.5 and NE_PM2. In various embodiments, the amount of peptoproducts a can vary as described under “Peptide Concentrations”, whereas the concentration of the NE may also vary, for example, from about 2 to about 50% v/w, from about 2 to about 40% v/w, about 2 to about 30% v/w, from about 3% to about 30% v/w, form about 3% to about 20% v/w, from about 3% to about 20% v/w, from about 4% to about 25% v/w, from about 4% to about 15%, from about 5% to about 20% v/w, from about 4.5% about 10%, from about 5% to about 20%, from about 5% to about to about 12% v/w, from about 5% to about 10% v/w, further for example, at least 1% v/w, at least 2% v/w, at least 3% v/w, at least 4% v/w, at least 5% v/w, at least 7% v/w, at least 9%, at least 10% v/w, at least 12% v/w, at least 15% v/w at least 20%, or more.

Rheological measurements were performed using MCR 302 rheometer (Anton Paar, Les Ulis, France) fitted with a 25 mm cone-plate geometry. The temperature was set at 25° C. The applied strain (γ %) was fixed at 0.5% within the linear viscoelastic regime on the basis of a previous amplitude sweep test. The empty hydrogels and nanocomposites apparent storage and loss moduli were measured by mean of frequency sweep tests over an angular frequency range of 100-0.0264 rad/s.

Rheology can be used to evaluate the average mesh size of the hydrogels [Karvinen J, Ihalainen T O, Calejo M T, Jönkkäri I, Kellomäki M. Characterization of the microstructure of hydrazone crosslinked polysaccharide-based hydrogels through rheological and diffusion studies. Mater Sci Eng C Mater Biol Appl. 2019 Jan. 1; 94:1056-1066. doi: 10.1016/j.msec.2018.10.048. Epub 2018 Oct. 17. PSID: 30423686.]. The average mesh size (ξ, nm), which is defined as the distance (Å) between the crosslinking points, can be calculated from the Eq. 5:

$$ε = {( \frac{{G}^{′} NA}{RT} )}^{- \frac{1}{3}} \qquad ( Eq . 5 )$$

where G′ is the storage modulus, NA is the Avogadro constant (6.022*10²³), R is the gas constant (8.314 J/K mol), and T is the temperature (310 K).

The crosslinking density of the hydrogels can also be evaluated. The crosslinking density (nₑ, mol/m³), which describes the number of elastically active junctions in the network per unit of volume, can be calculated from Eq. 6:

$$n e = {( \frac{{G}^{′}}{R T} )}^{- \frac{1}{3}} \qquad ( Eq . 6 )$$

We analyzed the data obtained by frequency sweep analysis with a cone/plate geometry of the empty hydrogels at the two different concentrations, 0.5% and 2.0% w/vol PS, and the respective nanocomposite contained at 5% w/vol (see above).

### Self-Assembling Peptides for Use with Nanocomposites

The rheological properties of self-assembling peptides PURASTAT (RADA16; (SEQ ID NO:1)), IEIK13 (SEQ ID NO:2), QLEL12 (SEQ ID NO:3) and KLD12 (SEQ ID NO:4)—each having unique physical and biochemical properties, are suited to the present invention, have been disclosed previously. PURASTAT is comprised of the synthetic peptide Ac-RADARADARADARADA-CONH₂ (SEQ ID NO:1), and potentially truncated fragments thereof, and is commercially supplied as a solution at 2.5% wt/vol in water (3-D Matrix, Ltd., Japan). The SAP IEIK13 (SEQ ID NO:2) shows different gelation characteristics when applied as a solution in vitro and in vivo when brought in to contact with biological fluids, such as blood, or in vivo-like conditions. Both these SAPs form viscous hydrogels with a nanofibrous matrix in a range of concentrations at about neutral pH. The SAPs disclosed in this application share this and other characteristics despite having different compositions.

PURASTAT is comprised of the amphiphilic self-assembling peptide RADA-16 (aspartic acid, arginine, alanine; (SEQ ID NO:1)) supplied sterile at 2.5% (wt/vol) in water (3-D Matrix). The solution has a pl of 7.2, pK1 of 1.79, and pK2 of 12.58 and exhibits different behaviour and properties at different pH values. At pH 2.2 PS is a viscous solution. Once the gel is touched/broken, the reassembly is slow because of strong repulsive (+) electrostatic interactions. At pH 2.5-4, PS forms a semi-rigid, viscous solution. The gel formation is triggered by hydrophobic and charge-charge interactions. Once the gel is broken, the reassembly is fast due to weak electrostatic interactions. Between pH 4 and 7.5 the self-assembling peptides form a rigid hydrogel. Nanofiber formation is due to hydrophobic and attractive charge-charge interactions.

Both RADA16 (SEQ ID NO:1) and IEIK13 (SEQ ID NO:2) have shown utility in promoting rapid hemostasis when applied to oozing biological tissues and, upon forming a hydrogel matrix integral with wounded tissue, promote normal healing over time rather than scar formation or lack of healing. As reported by Katsuyama et al. (Minimally Invasive Therapy & Allied Technologies 29(5): 283-292 (2020)), IEIK13 (called TDM-623 in that reference) forms a stiffer gel (i.e., has a higher storage modulus, G′) compared to RADA16 (TDM-621 in that reference) when exposed to physiological conditions which correlated with improved hemostasis when the product was applied to liver punch hole injuries in pigs. While reporting statistically significant improvement in hemostasis compared to RADA16, these authors also reported the absence of inflammatory cell infiltration due to the presence of the RADA16 or IEIK13 following application of the SAP solutions to the respective wounds in the short-term study. In a more recent study, IEIK13 ((SEQ ID NO:2); (called TDM-623 in that reference) was tested as a hemostatic agent administered endoscopically to oozing wounds created in the walls of the stomach and/or duodenum of pigs with apparent success, although no control group was included (Kubo et al., Endoscopic application of novel, infection-free, advanced hemostatic material: Its usefulness to upper gastrointestinal oozing (2021); doi.org/10.1002/deo2.25). As also found in the previously cited study, hemostasis was achieved in both heparinized and non-heparinized animals. It is noteworthy that, given the acidic pH in the stomach, application of IEIK13 still resulted in hemostasis of gastric bleeding.

The SAPs used in this invention are wholly synthetic and carry no risk of infection from animal-derived products. In addition, it has been reported in numerous studies that RADA16 and IEIK13 do not themselves promote an immune response or inflammation (e.g., Katsuyama et al.; Kubo et al.).

Other disclosures have detailed the rheological properties and gelation characteristics of RADA16 (SEQ ID NO:1) and other SAPs with the aim of applying the SAP solution directly to a tissue (see, for example, U.S. Pat. No. 10,654,893). In the present disclosure, PURASTAT (RADA16 (SEQ ID NO:1) supplied 2.5% wt/vol in water) is embedded with nanoemulsion (NE) particles having a hydrophobic core carrying either a fluorescent label or one of two hydrophobic drugs, Tofacitinib (TFC) or Curcumin (CCM), already used to treat inflammatory bowel disease (IBD), creating a nanocomposite shown herein to have improved therapeutic efficacy compared to either PURASTAT or drug-loaded NE particles alone when delivered by oral gavage in a mouse model of colitis. In preferred embodiments, peptide concentration prior to formation of the nanocomposite in the present invention is preferably within a range of range of about 0.05% to about 4% in solution or any other concentration listed under “Peptide Concentrations” below. Additionally, the dried nanocomposite gel is shown to rapidly rehydrate upon contact with water while maintaining the NEs intact within the nanofiber matrix. Oral administration of the dried nanocomposite enclosed within a pH-sensitive, time-released, or other capsule, has the potential to provide delivery of the nanocomposite to one or more areas of the gastrointestinal tract (GIT) where the capsule will dissolve, releasing the nanocomposite such that it rehydrates in the GIT or on association with the damaged or diseased tissue and releases an effective amount of the drug(s) carried by the NEs to treat the targeted area. As described, the SAP hydrogel component of the nanocomposite system assists in the healing of damaged or diseased tissue while at the same time encompassing the drug-loaded NEs thus providing sustained localized release of bioactive drugs that might otherwise be degraded before reaching their target.

The use of D-amino acid containing SAPs, including, RADA16 and IEIK13, as the hydrogel component of the nanocomposite system are included in embodiments of the present invention, particularly where a slower rate of in vivo degradation and/or resorption may be desirable. The appropriate peptide concentrations for RADA16, IEIK13, and all other peptides listed under “Peptide Concentrations” below.

In some embodiments of the present invention, where the SAP is KLD12 (SEQ ID NO:4). In some particular embodiments, where the SAP is IEIK13 (SEQ ID NO:2), peptide concentration prior to formation of the nanocomposite in the present invention is preferably within a range of range of about 0.05% to about 2.0% in water, given that beyond this concentration in water only the peptide may be too viscous to form a useful hydrogel (U.S. Pat. No. 10,654,893, see Table 1 therein) for a nanocomposite. However, when other components, such as NEs, are added to the IEIK13 (SEQ ID NO:2) solution and/or attached to the peptides, e.g., biologically active peptides and/or pharmaceuticals, the rheological properties of the solution may change. As disclosed in the following studies, however, PURASTAT (SEQ ID NO:1) did not show changes in rheological properties when loaded with NEs up to 10% wt/vol.

Peptide Concentrations—Rheological properties of peptide compositions as described previously (U.S. Pat. No. 10,654,893) may be controlled by selection of peptide concentration, for example as may be specifically preferred for a particular indication or use of compositions, through selection and/or adjustment of peptide concentration. For numerous SAPs, composition stiffness has been shown in vitro to increase substantially linearly with peptide concentration.) Any of the peptides recited in Table 2 can be used at concentrations while in non-swollen solution within a range of (all v/w) about 0.05% to about 5%, from about 0.05% to about 4%, from about 0.5% to about 4%, from about 0.5% to about 3.5%, from about 0.5% to about 3%, from about 0.5% to about 2.5%, from about 0.5% to about 2%, from about 0.5% to about 1.5%, or about 1%, about 2%, about 2.5%, about 3%, about 4%, about 5%.

In vitro, the rheological properties achieved at a particular peptide concentration vary depending on the identity of the peptide. For example, the storage modulus G′ of KLD12 (SEQ ID NO:4) 1.5% in water was found to be about 350 Pa similar to that of 2.5% RADA16 (SEQ ID NO:1) in water under the same test conditions. The storage modulus G′ of 1% IEIK13 (SEQ ID NO:2) in water (˜700 Pa) was found to be similar to that of 2.5% KLD12 (SEQ ID NO:4) in water and higher than that of 2.5% RADA16 (SEQ ID NO:1) in water (˜350 Pa) under the same test conditions (U.S. Pat. No. 10,654,893—Tables 3 and 3A). Overall, the order of rheological strength among these compositions was IEIK13 (SEQ ID NO:2)>KLD12 (SEQ ID NO:4)>RADA16 (SEQ ID NO:1), so a composition of IEIK13 (SEQ ID NO:2) showed greater rheological strength than did a composition of KLD12 (SEQ ID NO:4), which in turn showed greater rheological strength than did a composition of RADA16 (SEQ ID NO:1) when peptide concentration in water was the same in each case. Given the new results obtained by loading nanoemulsion particles (NEs) in RADA16, the previous characterization of various SAPs provides a reasonable expectation that the formulation of nanocomposites comprising different SAPs embedded with drug-loaded NEs will also show rheological properties similar to or the same as the plain SAPs.

Accordingly, in some embodiments of the invention, the SAPs comprise a sequence of amino acid residues conforming to one or more of Formulas I-IV:

((Xaa^{neu}−Xaa⁺)×(Xaa^{neu}−-Xaa⁻)y)n (I)

((Xaa^{neu}−Xaa⁻)×(Xaa^{neu}−-Xaa⁺)y)n (II)

((Xaa⁺−Xaa^{neu})×(Xaa⁻−Xaa^{neu})y)n (III)

((Xaa⁻−Xaa^{neu})×(Xaa⁺−Xaa^{neu})y)n (IV)

Xaa^{neu} represents an amino acid residue having a neutral charge; Xaa⁺ represents an amino acid residue having a positive charge; Xaa⁻ represents an amino acid residue having a negative charge; x and y are integers having a value of 1, 2, 3, or 4, independently; and n is an integer having a value of 1-5.

The above and other peptides suitable in methods of the invention are similar to RADA16 and includes the following listed in Table 2 and modified peptides and peptidomimetics listed further below (all with appropriate N-terminal carboxylation and C-terminal amidation of the peptides) and any of the appropriate concentrations listed under “Peptide Concentrations” above.

TABLE 2
Examples of Peptides Useful in the Methods of
the Invention

| | |
|---|---|
| SEQ ID NO: 1 | RADARADARADARADA |
| SEQ ID NO: 2 | IEIKIEIKIEIKI |
| SEQ ID NO: 3 | QLELQLELQLEL |
| SEQ ID NO: 4 | KLDLKLDLKLDL |
| SEQ ID NO: 5 | RATARAEARATARAEA |
| SEQ ID NO: 6 | ADARADARADARADAR |
| SEQ ID NO: 7 | RAEARAEARAEARAEA |
| SEQ ID NO: 8 | EARAEARAEARAEARA |
| SEQ ID NO: 9 | RVDVRVDVRVDVRVDV |
| SEQ ID NO: 10 | RLDLRLDLRLDLRLDL |
| SEQ ID NO: 11 | RIDIRIDIRIDIRIDI |
| SEQ ID NO: 12 | RFDFRFDFRFDFRFDF |
| SEQ ID NO: 13 | AEARAEARAEARAEAR |
| SEQ ID NO: 14 | KADAKADAKADAKADA |
| SEQ ID NO: 15 | KIDIKIDIKIDIKIDI |
| SEQ ID NO: 16 | KIEIKIEIKIEIKIEI |
| SEQ ID NO: 17 | IDIKIDIKIDIKI |
| SEQ ID NO: 18 | IEIRIEIRIEIRI |
| SEQ ID NO: 19 | LELKLELKLELKL |
| SEQ ID NO: 20 | FEFKFEFKFEFKF |
| SEQ ID NO: 21 | KLELKLELKLEL |
| SEQ ID NO: 22 | FEFRFEFRFEFRF |
| SEQ ID NO: 23 | YEYKYEYKYEYKY |
| SEQ ID NO: 24 | WEWKWEWKWEWKW |

In some embodiments of this invention, the peptoproducts further comprise an amino acid sequence that interacts with the extracellular matrix, wherein the amino acid sequence anchors the SAPs to the extracellular matrix (for example, RGD).

In other embodiments, the amino acid residues in the SAPs can be (synthetic or not animal derived) naturally occurring or non-naturally occurring amino acid residues. Naturally occurring amino acids can include amino acid residues encoded by the standard genetic code while non-naturally occurring amino acid include non-standard amino acids (e.g., amino acids having the D-configuration instead of the L-configuration or combinations of D- and L-amino acids), as well as those amino acids that can be formed by modifications of standard amino acids (e.g., pyrolysine or selenocysteine). Suitable non-naturally occurring amino acids include, but are not limited to, D-alloisoleucine(2R,3S)-2-amino-3-methylpentanoic acid, L-cyclopentyl glycine (S)-2-amino-2-cyclopentyl acetic acid. In some embodiments, the SAPs used in the method of invention comprise only naturally occurring amino-acids, or only unnaturally occurring amino acids (such as D-amino acids, e.g., RADA16 or IEIK13 comprised of D-amino acids); or combination of both D- and L-amino acids. RADA16 comprised of D-amino acids, as well as other SAPs, could be used in the methods of the invention, to potentially reduce the in vivo degradation of the hydrogel matrix thereby increasing the retention time of the hydrogel at the site of adhesion, which, in turn, could affect the retention of solutes (e.g., drugs) within the gel matrix, ingrowth of tissue into the matrix and tissue healing.

In other embodiments, another class of materials that can self-assemble and mimic the SAPs are peptidomimetics. Peptidomimetics, as used herein, refers to molecules which mimic peptide structure. Peptidomimetics have general features analogous to their parent structures, polypeptides, such as amphiphilicity. Examples of such peptidomimetic materials are described in Moore et al., Chem. Rev. 101(12), 3893-4012 (2001). The peptidomimetic materials, used in the invention, can be classified into four categories: α-peptides, β-peptides, γ-peptides, and δ-peptides. Copolymers of these peptides can also be used. Examples of α-peptide peptidomimetics include, but are not limited to, N,N′-linked oligoureas, oligopyrrolinones, oxazolidin-2-ones, azatides and azapeptides. Examples of β-peptides include, but are not limited to, β-peptide foldamers, α-aminoxy acids, sulfur-containing β-peptide analogues, and hydrazino peptides. Examples of γ-peptides include, but are not limited to, γ-peptide foldamers, oligoureas, oligocarbamates, and phosphodiesters. Examples of δ-peptides include, but are not limited to, alkene-based δ-amino acids and carbopeptoids, such as pyranose-based carbopeptoids and furanose-based carbopeptoids.

In certain embodiments, the SAP is AC5®, AC5-V®, AC5-G™ or TK45, also known as AC1, made by and available from Arch Therapeutics, Inc. (see www.archtherapeutics.com). Each of these self-assembling peptides, and others disclosed herein, are capable of forming a hydrogel when applied to a biological tissue (e.g., in situ) at about neutral pH. Generally, the SAP concentration in water will range from about 1% to about 5% weight/volume although this range is not exclusive. For example, the aqueous concentration at which IEIK13 (SEQ ID NO:2) peptides form a hydrogel matrix when exposed to physiologic conditions is generally between about 0.5 and about 2.5%. However, self-assembly (or not) of this and other peptides in aqueous environments depends on numerous variables including pH, ionic concentration, the concentration and composition of the SAP itself, the type of ions present, and numerous other factors. Data characterizing and comparing the properties of RADA16 (SEQ ID NO:1), IEIK13 (SEQ ID NO:2) and KLDL (SEQ ID NO:4) under different conditions can be found in U.S. Pat. No. 10,654,893; the entire contents of which are incorporated herein by reference.

### EXAMPLES

### Example 1. Rheological Characterization

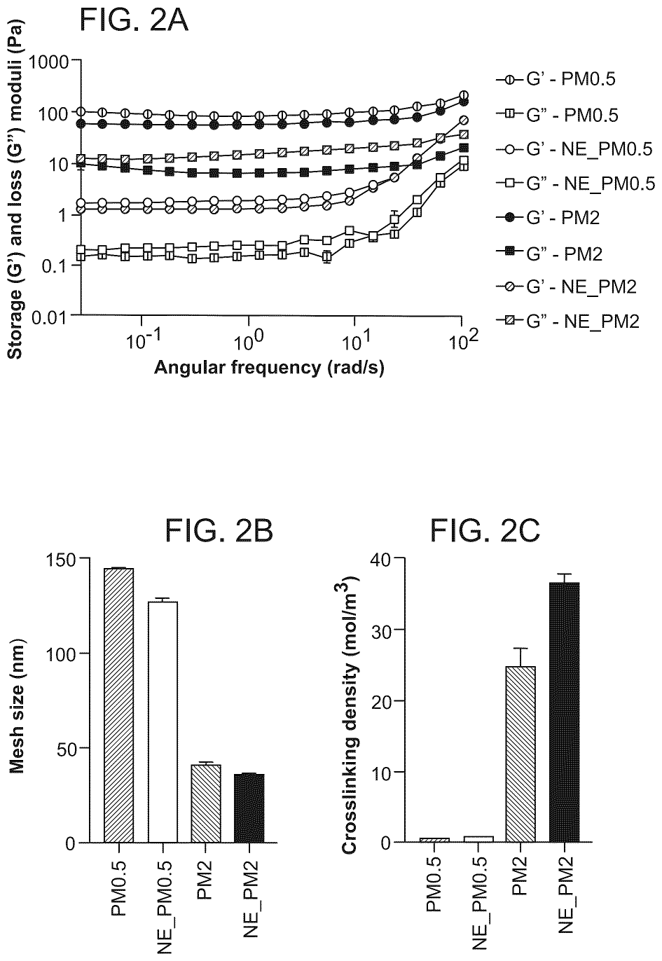

The frequency sweep measurements were performed at variable frequencies at the linear viscosity region (LVR) strain amplitude determined by amplitude sweep measurements. As shown in FIG. 2A, for all hydrogel composites, G′ was higher than G″ (elastic response is stronger than the viscous one) and independent of frequency. G′ was also parallel to G″. The ratio of G″ to G′ (tan δ) was <0.2 for all hydrogels, and therefore their structures were considered to be strong. Due to these observations, these hydrogels were considered to be stable and strongly crosslinked gels. The G′, G″ and G* of the hydrogels presented in Table 2 were determined at 1.27 rad/s. The results showed that the G′ (and G*) of the hydrogels was lower at the smallest concentration. Table 1 above shows a comparison of the NE used in the present Examples.

TABLE 3
Hydrogel parameters determined based on the rheological frequency sweep
analysis. Storage moduli (G’), loss moduli (G”), complex moduli (G*), average mesh
sizes ( [symbol] ), and average crosslinking densities (nₑ). Low crosslinking density corresponds
with big pores (5 to 200 nm)

| | | | | | Average | Crosslinking |
|---|---|---|---|---|---|---|
| | | | Loss | | mesh size, [symbol] | Density, nₑ |
| Sample | G’ (Pa) | G” (Pa) | Tangent, T | G* (Pa) | (nm) | (mmol/m³) |
| PM0.5 | 1.36 ± 0.03 | 0.17 ± 0.01 | 0.12 ± 0.01 | 1.37 ± 0.03 | 144.65 ± 1.03 | 0.55 ± 0.01 |
| NE_PM0.5 | 1.99 ± 0.16 | 0.26 ± 0.00 | 0.13 ± 0.01 | 2.01 ± 0.16 | 127.39 ± 3.60 | 0.80 ± 0.07 |
| PM2 | 61.37 ± 11.95 | 7.05 ± 1.11 | 0.11 ± 0.01 | 61.77 ± 12.0 | 40.63 ± 2.86 | 24.76 ± 4.82 |
| NE_PM2 | 90.38 ± 6.42 | 16.25 ± 0.34 | 0.18 ± 0.01 | 91.83 ± 6.35 | 35.71 ± 0.87 | 36.46 ± 2.59 |

Microstructure plays an essential role in the control of hydrogel properties. It is also an important factor when cells or drugs are encapsulated inside the hydrogel. The microstructure of PURASTAT and of the nanocomposites were evaluated by using a rheology-based method. The average mesh sizes (ξ) of the hydrogels were calculated using Eq. 5. The ξ of the empty hydrogels was of 144.65±1.03 nm and of 40.63±2.86 nm, for PS0.5 and PS2 respectively, whereas the one of the NE-embedded hydrogels was 127.39±3.60 nm and 35.71±0.87 nm for NE_PM0.5 and NE_PM2, respectively. These results are shown in FIG. 2B. The ξ of the hydrogels decreased when the polymer concentration increased, while the presence of NEs did not show any impact on the hydrogels' rheological structure. The crosslinking densities (nₑ) of the hydrogels were calculated using Eq. 6. The calculated parameters are shown in Table 2 and illustrated in FIG. 2C. The nₑ was of 0.55±0.01 and 24.76±4.82 mmol/m³ for PS 0.5% and PS 2%, respectively. The nanocomposites showed similar results, 0.80±0.07 and 36.46±2.59 for NE_PM0.5 and NE_PM2, respectively. The nₑ of the hydrogels decreased when the concentration of the hydrogel decreased.

### Example 2. Drying of the Nanocomposite

The system was converted into a dried solid powder using a freeze-drying technique. Based on the slightly increased size of the NEs after being loaded into the PS hydrogel compared to the size measured before being loaded, PURASTAT appeared to create a shell which surrounded the NEs. Also, no additional cryoprotectant was needed. The cake obtained was white, smooth and compact. Resuspension in water was easy and re-gelation was immediate. NEs maintained their properties, with no significant variation in size. The higher PDI value could be due to the presence of PS in solution (Table 4).

TABLE 4
Characterization of the NE doped PM hydrogel
before freeze-drying and after rehydration

| | | | Size Before | | Size After | |
|---|---|---|---|---|---|---|
| Sample | PM % | NE % | (nm) | PDI | (nm) | PDI |
| PM0.5%NE0.5% | 0.5 | 0.5 | 108.10 ± 0.53 | 0.26 | 131.03 ± 16.1 | 0.48 |
| PM0.5%NE1% | 0.5 | 1 | 108.33 ± 0.93 | 0.26 | 141.03 ± 20.2 | 0.65 |
| PM0.5%NE2.5% | 0.5 | 2.5 | 107.50 ± 1.31 | 0.25 | 179.3 ± 10.89 | 0.52 |
| PM0.5%NE5% | 0.5 | 5 | 107.23 ± 0.85 | 0.25 | 198.57 ± 11.29 | 0.51 |
| PM0.5%NE12.5% | 0.5 | 12.5 | 111.43 ± 2.86 | 0.25 | — | — |

For the samples with NE percentage of 0.5 and 1 the dried nanocomposite appeared as white compact cakes. Samples having 2.5% and 5% NE became white, brittle compact cakes when dried. All of these samples rehydrated easily with addition of water and gentle shaking by hand and immediately reformed a hydrogel. The samples with 12.5% NE, by comparison, formed a white compact cake on being freeze-dried but did not rehydrate.

### Example 3. Circular Dichroism Characterization of Nanocomposite Formulation for In Vivo Studies

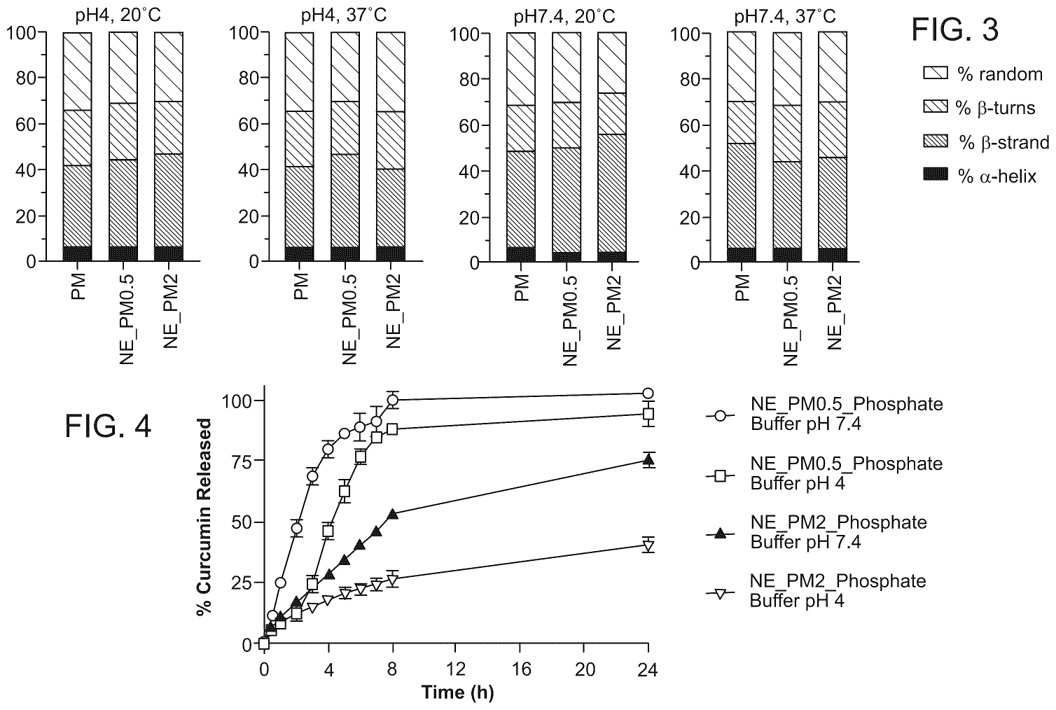

CD experiments were used to understand if the incorporation of nanoparticles would adversely affect the initial self-assembly of the peptide through analyzing the β-sheet peak, which is indicative of nanofiber formation (Lu, L. & Unsworth, L. D. PH-Triggered Release of Hydrophobic Molecules from Self-Assembling Hybrid Nanoscaffolds. Biomacromolecules 17, 1425-1436 (2016)). Control experiments were conducted using CD characterization of nanoparticles without the presence of the peptide where no response as a function of wavelength was observed. The peptide controls exhibited a typical β-sheet structure (minimum at 217-218 nm, maximum at 195-206 nm) for systems without nanoparticles. This secondary structure content was further quantified using CDNN software, yielding to the percentages shown in FIG. 3. As seen in the Figure, no clear difference in terms of secondary structure was detected upon incubation of PS with the NEs.

### Example 4. Model Drug Release Study

After the nanocomposites' characterization, the ability of a drug-loaded NE to be released from the hydrogel was studied. Curcumin is an active principally characterized by low aqueous solubility, poor stability in the body fluids, high rate of metabolism, rapid clearance, reduced absorption in the gastrointestinal tract (GIT) and limited bioavailability. These are common characteristics among many of the small molecules conventionally used for the treatment of many diseases (Yavarpour-Bali, et al., Curcumin-loaded nanoparticles: A novel therapeutic strategy in treatment of central nervous system disorders. International Journal of Nanomedicine vol. 14 4449-4460 (2019)). Curcumin (CCM) was loaded in the nanoemulsion in order to follow its release from the nanoparticles in the PS nanocomposite. The NE release from NE_PM0.5 and from NE _PM2 were tested in phosphate buffers at pH 4 and 7.4, respectively. One gram of the nanocomposite in the hydrogel form were weighed in a vial and 4 mL of the respective buffer was added on top. At scheduled timepoints, the supernatant was removed and substituted with the same volume of fresh buffer. The amount of released curcumin, proportional to the amount of released NE, was quantified by RP-HPLC.

The cumulative results of these studies are shown in FIG. 4. The results of this model study underline the effect of both peptide concentration, and pH, on the drug release. The higher the PS concentration, the slower the release. These results agree with the model study rheological studies that showed that in the presence of bigger meshes and a lower crosslinking density for PS0.5, such that the NEs are able to release the CCM loaded in them faster than in PS2, which is characterized by a higher density of crosslinking and a lower mesh size. On the other hand, the pH also affects the hydrogel behavior. The isoelectric point of the RADA16 (SEQ ID NO:1) peptide is 7.2, meaning that at lower pH the mesh and the structure of the hydrogel are intact, while the shift to higher pH leads to a change in its drug release characteristics.

### Example 5. In Vivo Model of Colitis

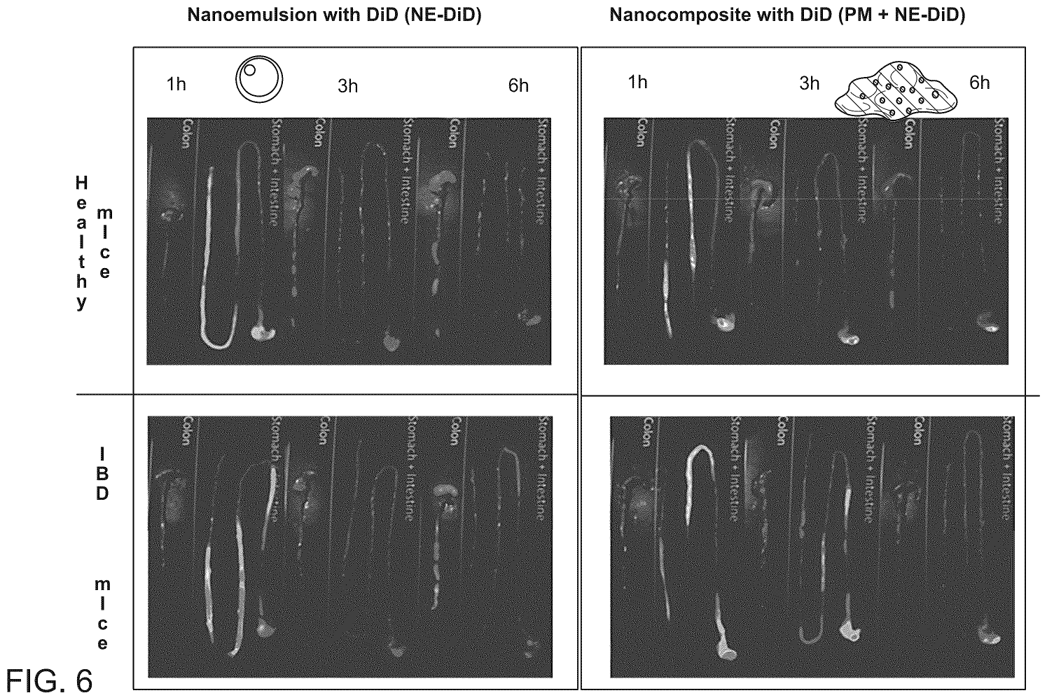

In the first stage of these in vivo studies, we evaluated the experimentally determined data related to DSS-induced colitis in the mice such that the colitis induced in the mice was not so extreme as to be untreatable. As shown in FIG. 6, the mice were divided into four groups. In G1 and G2 the mice were given 3% DSS in drinking water for 4 days. The G1 mice and G2 mice were then sacrificed at day 7 and day 14, respectively. The G3 and G4 mice were given 3% DSS in drinking water for 7 days and were then sacrificed at day 7 and day 14, respectively. From the beginning of the study until sacrifice, all the mice were scored on the following factors:

TABLE 5
Disease activity index (DAI) score is used to evaluate the DSS-induced
colitis. DAI index was calculated as total score (body weight decrease +
stool consistency + rectal bleeding) divided by 3.

| Score | Weight loss % | Stool consistency | Hematochezia |
|---|---|---|---|
| 0 | None | Normal feces | No blood |
| 1 | 0-5 | Mild loose stool | |
| 2 | 5-10 | Loose wet stool, diarrhea | Presence of blood |
| 3 | 10-20 | Watery diarrhea | |
| 4 | >20 | Diarrhea with blood | Visible bleeding |

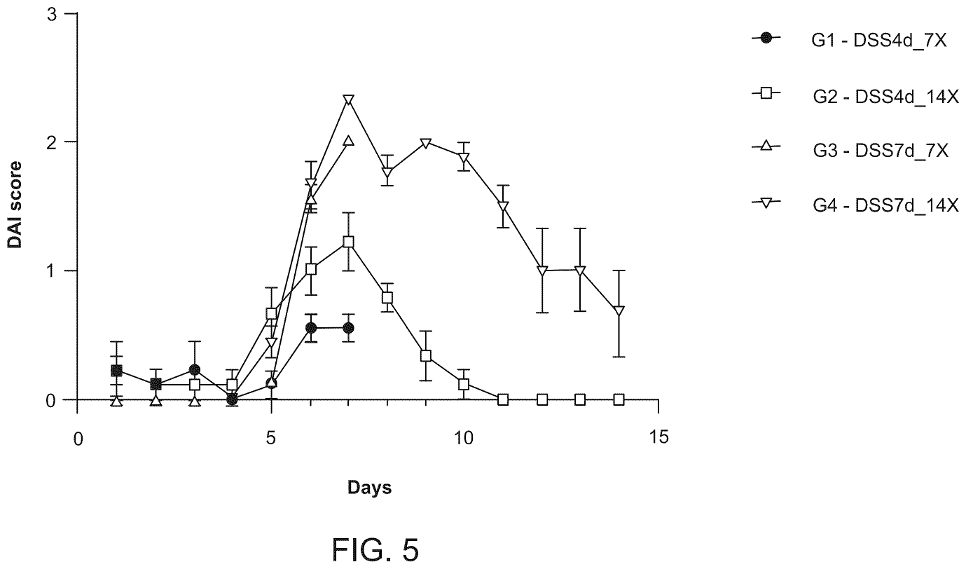

The experimentally observed DAI scores for the four groups of mice are plotted in FIG. 5. These results showed that an administration of 3% v/v DSS in drinking water over a period of 7 days led to too severe colitis induction, while administration for 4 days was followed by a slow and complete recovery of the animals. In fact, in these studies it was important not to induce an extreme condition that would be too difficult to treat. For this reason, an incubation time with DSS for 4 days was chosen for the next studies, followed by treatment/recovery until day 10 that showed good progress towards recovery in terms of macroscopic and histological evaluations in stage 1.

### Example 6. In Vivo Distribution of Fluorescent Dye-Loaded PS_NE in the Gastrointestinal Tract of Mice with DSS-Induced Colitis

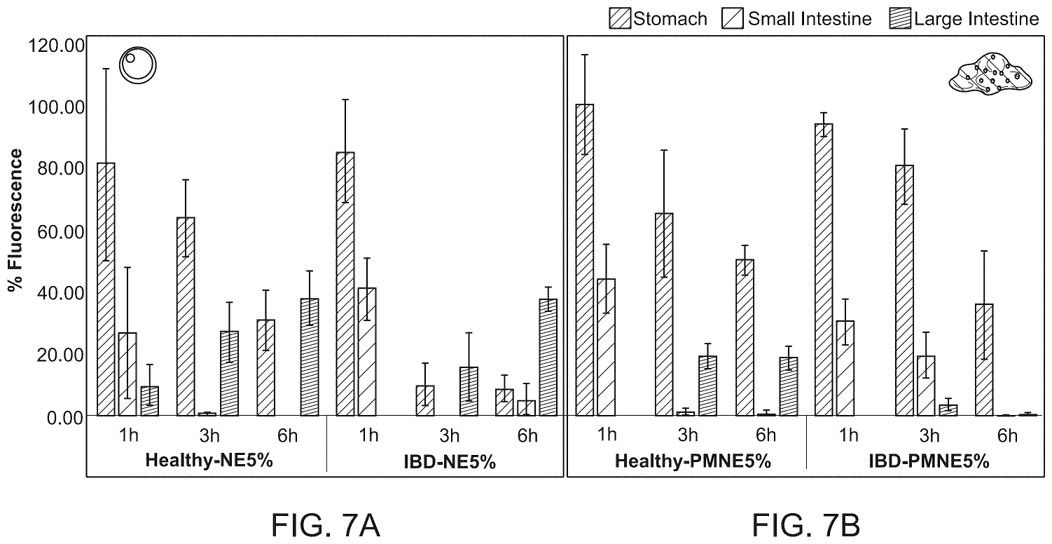

In this example, 3% DSS in drinking water was given to the IBD mice for 4 days, while the control healthy mice received normal water. At the end of the 4^{th} day, both groups of mice received 1,1′-dioctadecyl-3,3,3′,3′-tetramethylindodicarbocyanine, 4-chlorobenzenesulfonate salt (DiD) loaded NE alone (NE), or the nanocomposite containing the DiD-NE (DiD-NEPM), by oral gavage, mimicking the first treatment that the mice would receive in the efficacy study to be described. The mice were sacrificed at scheduled time points of 1, 3, 6, and 24 hours and their gastrointestinal tracts were extracted for further studies of the fluorescence distribution in the GIT. The last point at 24 hours—not shown—did not display any fluorescence. Fluorescence images of the tissues extracted at the earlier time points are shown in FIG. 6. Semiquantitative analysis of the images shown in FIG. 6 using the software Wasabi is shown in FIG. 7. In addition, qualitative fluorescence obtained by LSFM (light sheet fluorescence microscopy) after tissue clarification was observed in tissue from two of the IBD mice, one that received NE and one that received NEPM. Both were sacrificed at 3 hours after oral gavage.

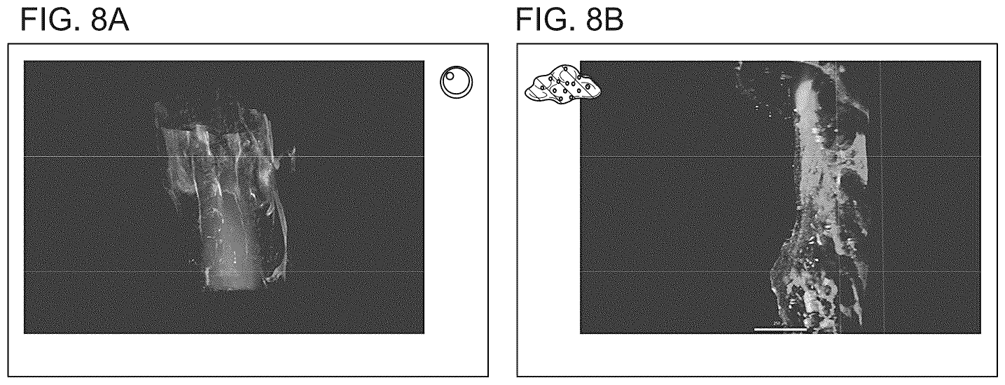

The images shown in FIG. 8 are particularly striking. For these images the upper section of the colon of IBD mice which received the NE-DiD in solution (A) or the PS-NE-DiD (B) by oral gavage was collected and fixed in PFA 4% v/v for 24 hours before being transferred into PBS, pH 7.4 for the qualitative visualization of the preferential accumulation of the dye in the tissue. X-clarity tissue clearing system was used to clarify the tissues and the fluorescence of the particles in the colon tissues ex-vivo was observed using light sheet fluorescence microscopy. FIG. 8A shows very little fluorescence while FIG. 8B—derived from an IBD mouse treated with PS loaded with DiD-NE—shows fluorescence from DiD distributed throughout most of the tissue sample. This observation shows that the inflamed colonic tissue takes up the nanocomposite NE particles very well while the tissue uptake of NE-DiD particles is very limited. Although only two mice were compared in this study, again the results are striking.

### Example 7. Efficacy of Drug-Loaded Nanocomposite System in Treating DSS-Induced Colitis

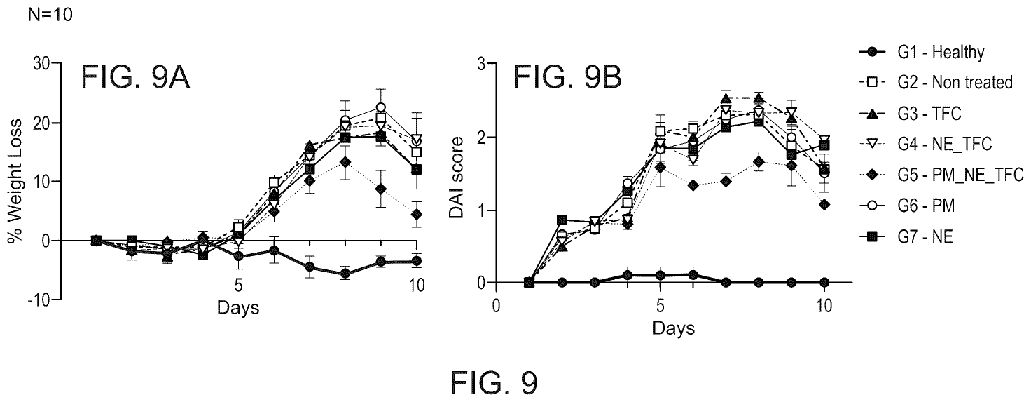

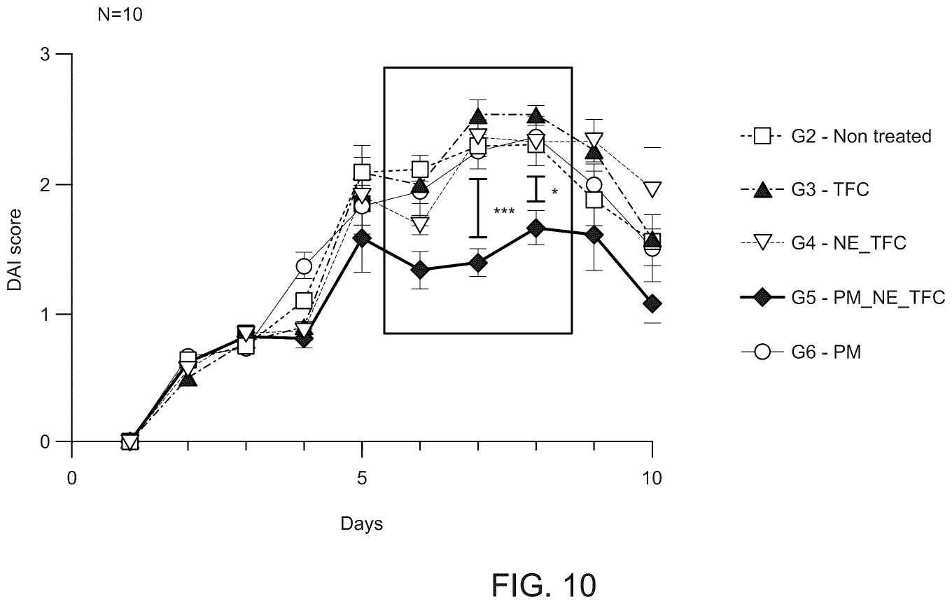

Efficacy of drug-loaded nanocomposites compared to a variety of controls was tested using mice of the same strain used in Example 5. Based on the results from the Stage 1 in vivo studies already described, all mice (except for the G1 healthy control group) were given 3% v/v DSS in drinking water for 4 days. After 4 days all mice were given normal drinking water (i.e., no DSS). After Day 4, the DSS-treated mice were orally administered different components of the nanocomposites with Tofacitinib (TFC)-loaded NE, as indicated in Table 4, by oral gavage on days 5, 7 and 9. The condition of the mice was observed as in Example 5/Stage 1 and scored by the criteria in Table 5. FIG. 9A illustrates the percent weight loss of the mice in each group over time and FIG. 9B plots the DAI score. FIG. 10 (derived from FIG. 9B) illustrates more clearly that a statistically significant improvement in DAI score for the IBD mice administered PSNE with TFC was seen at days 7 and 8 relative to the other IBD mice groups. A number of the mice in each group were sacrificed on days 5, 7 and 10 and tissues were then extracted for further analyses.

In FIG. 9A, one can observe the percentage of weight loss for all the groups involved in the study. G1 did not show any loss of weight, with a little augmentation over time. All the other groups showed a loss of weight beginning from day 5, when the DSS effect started to display macroscopically. Following the DAI data, G6 is the only one that also in terms of only body weight loss showed better results compared to the other groups. In FIG. 9B, one can observe the DAI score obtained for all the groups involved in the study. G1 showed a low score, while all the other groups followed a similar behavior profile, with a peak around day 7 followed by a slow remission. The only group that displayed a better profile was the G5, in which a little reduction of the DAI score was detected after the administration of the first treatment at day 5. This might be due to the slower and prolonged release not only of the drug from the nanosystem, but mainly of the nanosystem from the hydrogel.

TABLE 6
Stage 2 In Vivo studies of efficacy of
Tofacitinib-loaded NE-PM nanocomposite

Group 1 (G1): Healthy mice (n = 5), negative control
Group 2 (G2): DSS-induced colitis mice (n = 15), positive control
Group 3 (G3): DSS-induced colitis mice (n = 10), oral gavage with TFC
Group 4 (G4): DSS-induced colitis mice (n = 10), oral gavage with TFC-
loaded NE (TFC_NE)
Group 5 (G5): DSS-induced colitis mice (n = 10), oral gavage with TFC-
loaded NE embedded in PS (PM_NE_TFC)
Group 6 (G6): DSS-induced colitis mice (n = 10), oral gavage with empty
PS
Group 7 (G7): DSS-induced colitis mice (n = 10), oral gavage with empty
NE

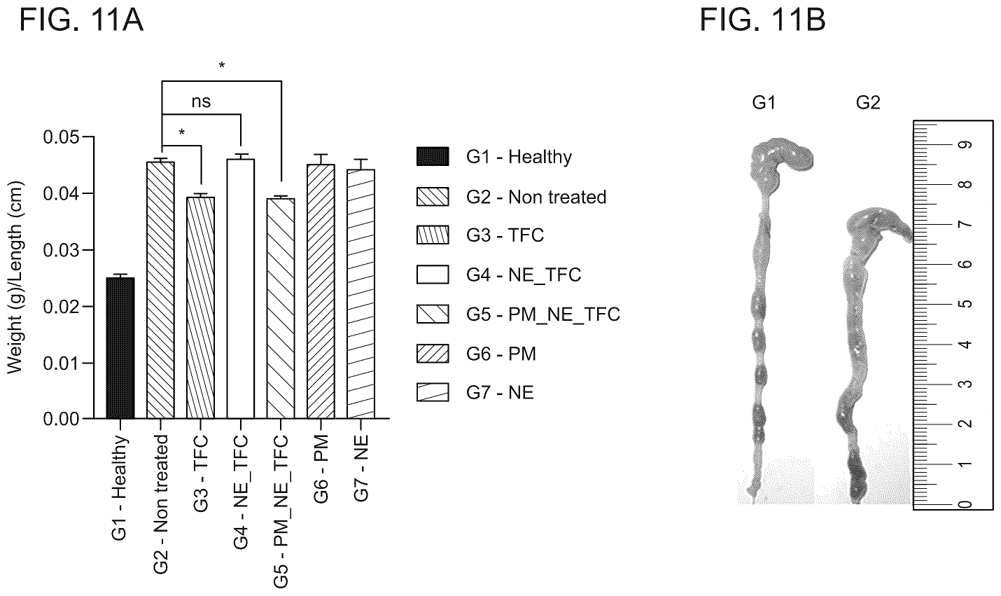

As shown in FIG. 11, a gross analysis (used in the prior art) was used by dividing the weight of each colon extracted from each mouse (g) divided by its length (cm) as shown for example in FIG. 11B. This data, plotted in FIG. 11A, shows a statistically significant improvement (i.e., lower g/cm) relative to G2 untreated IBD mice for G3—treated with plain TFC, and G5-PS_NE_TFC mice.

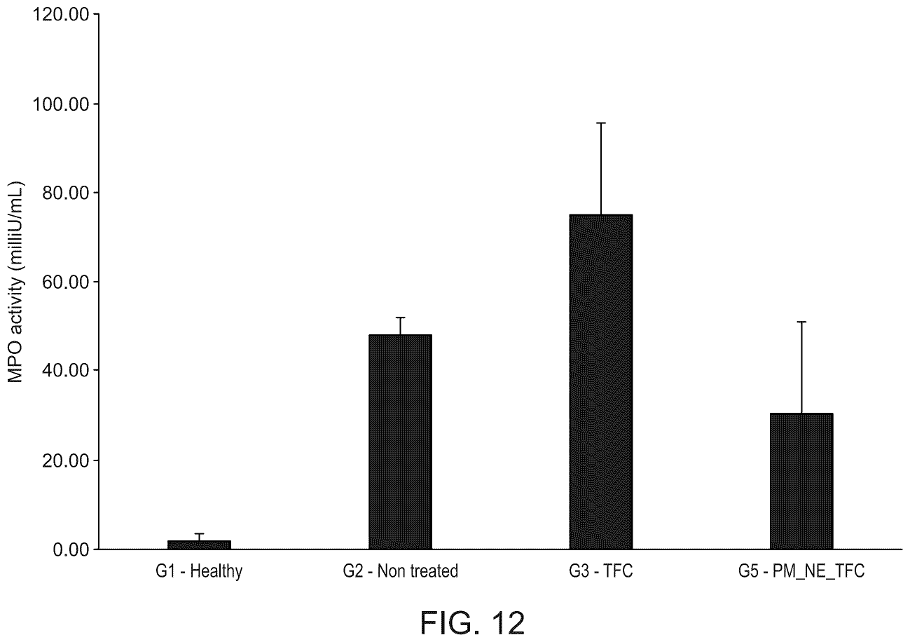

In FIG. 12, the results from quantification of Myeloperoxidase (MPO) activity as an indicator of neutrophil infiltration in the colonic mucosa are shown. The method as described here was used: www.sigmaaldrich.com/FR/fr/product/sigma/mak068?gclid=Cj0KCQiA15yNBhDTARIsA Gnwe0U8JudIWDIwyCYqrvusn2PQB3WnPk-ec3mxqu_5eAp6vwAYiItUdmYaAtC3EALw_wcB These results indicate that that MPO activity was lower in colonic tissue from IBD mice treated with PS_NE_TFC particularly when compared the G3 mice treated with plain TFC.

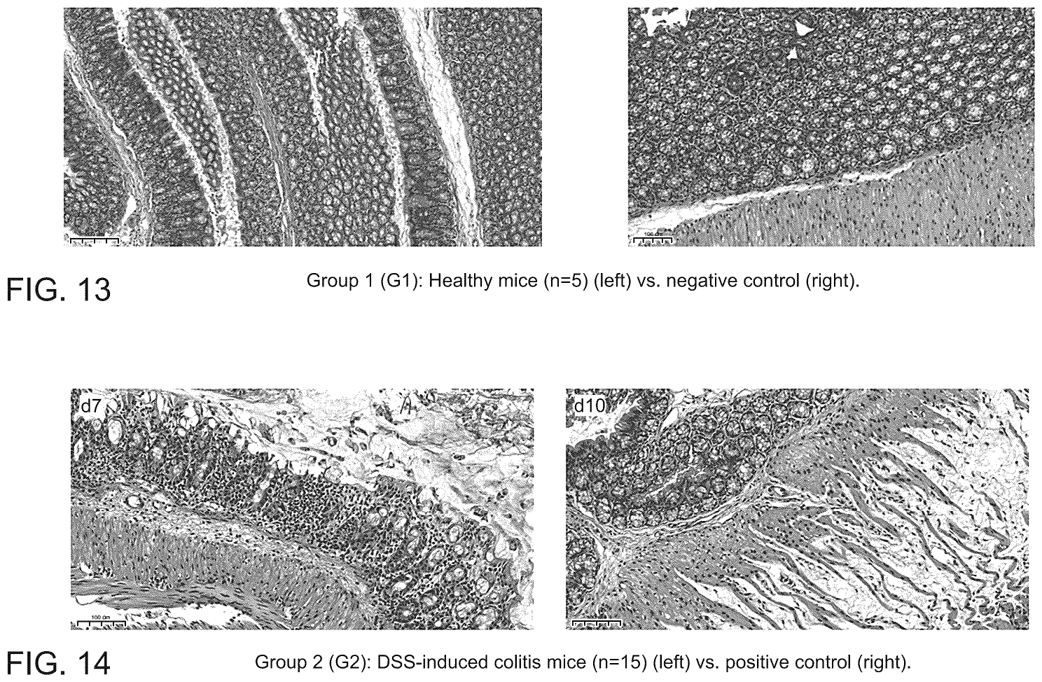

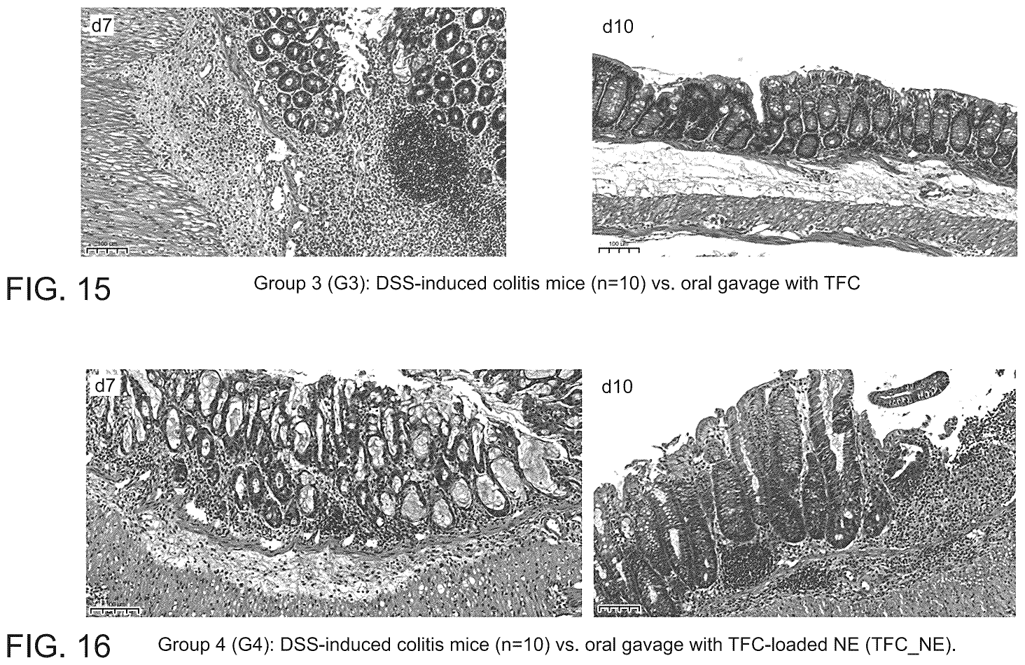

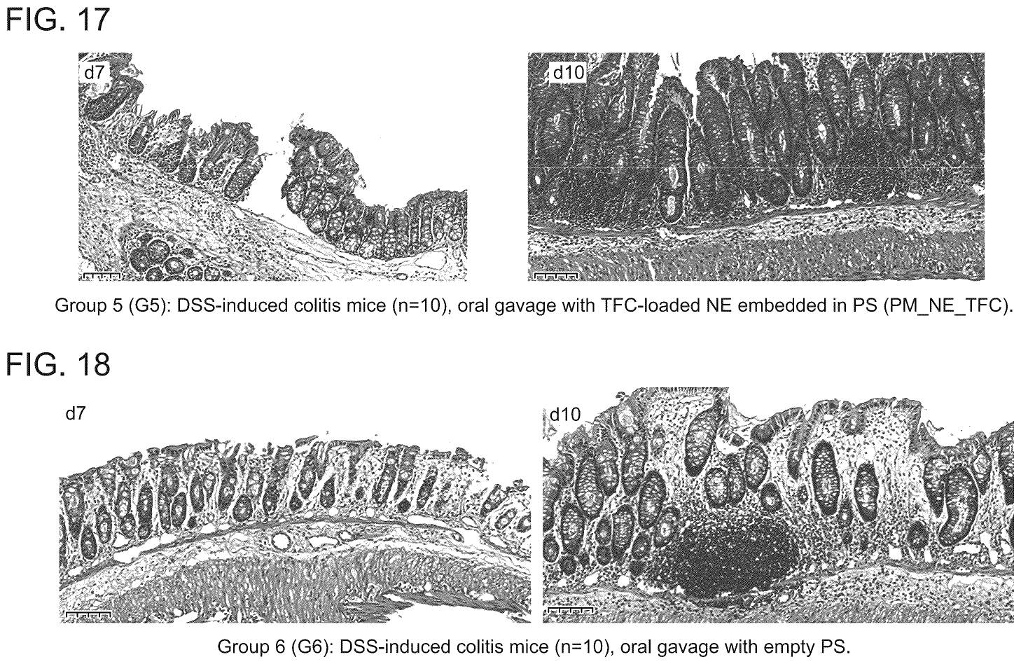

Exemplary histological results of colon tissue stained with HPS are shown in FIGS. 13 through 18 for mice from the different groups as indicated.

Macroscopic results from the study are plotted in FIG. 9. As shown more clearly in FIG. 10, the mice treated with TFC-loaded nanocomposite showed a statistically significant improvement in DAI score relative to the other treated groups on day 7 and day 8 of the study.

### Example 8. Comparison of Biodistribution in Healthy and IBD-Induced Mice

To assess the ability of the NEs or that of the hybrid system PS-NEs to be retained in the inflamed and healthy GIT, the nanoparticle was loaded with the fluorescent dye DiD (1,1′-Dioctadecyl-3,3,3′,3′-Tetramethylindodicarbocyanine, 4-Chlorobenzenesulfonate Salt) and analyzed by near-infrared fluorescence imaging following oral gavage. Observations were made at 1, 3, and 6 h of day 5, after 4 days of colitis induction using DSS 3% w/v (as defined in the previous study). Time points were chosen according to previous studies considering that mice have a total GI transit time of about 6 hours and that the majority of the intestinal content is located in small intestines and cecum after 3 hours (Rosso A., Andretto V., et al. Nanocomposite sponges for enhancing intestinal residence time following oral administration. J. Control. Release (2021) 333: 579-592). In order to perform a semi-quantitative analysis of the fluorescent dye distribution, organs were harvested and ex vivo images were taken as shown, for example, in FIG. 8. As shown in FIG. 9, the images collected at the different time points were processed to extract different information on residence time, targeting ability and potential toxic effect of the formulations.

As illustrated in FIG. 6 and analyzed, the biodistribution of NE in healthy animals shows a progressive passage from the stomach to the rest of the intestinal area, with a relevant fluorescence in the colon at 3 and 6 hours, a sign of progressive elimination of a detectable amount of fluorophore. In the case of the NE biodistribution in IBD mice the overall elimination from the stomach is faster and the amount of residual DiD in the GIT at 6 h is centred in the colon.

For administration of the hybrid composite, as shown in FIG. 7B, we observed a similar trend indicating fluorophore accumulation in the stomach over time, both in healthy and IBD mice. The main difference between the two sets of animals is preferential accumulation of detectable fluorescence in the small intestine for IBD mice, while for the healthy ones the passage through the small intestine appears to be faster, as we detected stronger fluorescence intensity in the colon.

### Example 9. Functional Effects of Self-Assembling Peptide Alone

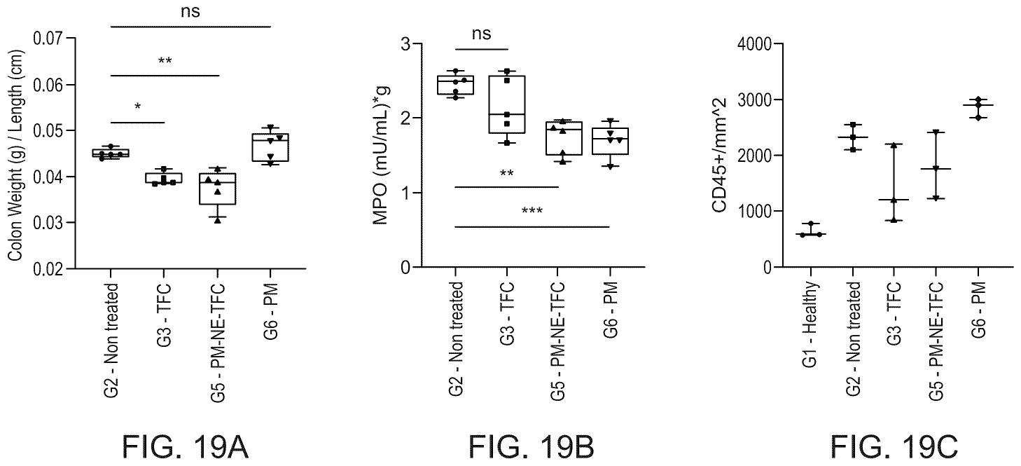

Group G6 samples described in Example 6 (DSS-induced colitis mice (n=10), oral gavage with Self-Assembling peptide alone) were further analyzed. FIG. 19A shows colon weight/colon length ratio average per group of mice (n=5). Similar to results obtained for weight loss, Group G6 displayed a significantly better profile, in which a reduction of the DAI score was detected since the administration of the first treatment, and reached the maximum effect at the peak of the profile (days 7 and 8). MPO enzymatic activity, as an indicator of granulocyte infiltration, was measured and the results are shown in FIG. 19B. The nanocomposites showed a significantly reduced activity of MPO, reduction that is maintained when animals are treated with only PS, while even if a slight improvement was obtained, the treatment with the free drug displayed a high intraindividual variability.

The treatment with TFC alone (G3) led to a considerable amelioration of the epithelium and muscularis structure, and to a reduced infiltration of lymphocytes, that was even more accentuated when the mice received the treatment double encapsulated in the nanocomposite structure (G5). The group treated with the hydrogel alone (G6) did not show a significative reduction of CD45+ cells infiltration as shown in FIG. 19C, even if a considerable improvement with respect to G2 was observed, especially in the architecture recovery.

When compared with free TFC treatment (G3), the administration of PM-NE-TFC (G5) greatly decreased both the activity and the density of neutrophils, resulting in a decrease of the myeloid cell population. The reduced effect on macrophages is possibly the result of the changes in the relative percentage of the different cell populations due to the strong reduction of granulocytes as also demonstrated by MPO analysis.

The total amount of T cells (CD3⁺) is strongly reduced by the presence of the drug, and it is maintained when PM alone is administered. Nevertheless, when analyzed as relative amount of total immune cells (CD45⁺ cells) we did not observe significant changes in the frequency of either CD8 or CD4 T cells, except an increase of CD4+ T cells in the LP of animals administered with PM only. Both TFC and PM treatments significantly reduced the proportion of both Th1 and Th17 proinflammatory but did not affect the Th2 or Treg populations. PM alone shows similar ability in reducing the proportion of both Th1 and Th17 proinflammatory populations compared to TFC in both free and encapsulated form.

These phenomena need further investigation, but since the involvement of T cells is not required for the initial development of DSS-induced colitis and represents a consequence of the primary inflammation, it is possible that PM role in reducing inflammatory cytokine production can interfere with the recruitment and differentiation of Th1 and Th17 populations consequently reducing or even preventing the exacerbation the innate immune response due to Th1 cytokines and the main product of Th17 population IL17A. Moreover, the efficacy of TFC is preserved when administered in the nanocomposite form. Not surprisingly, PM showed interesting anti-inflammatory properties that can be exploited to improve the therapeutic efficacy of the pharmacologic therapy as well as favoring the mucosal healing process.

The interest in the developed nanocomposite made of PM and NE-TFC relies on the synergistic effect due to the co-administration in a single dose of the therapeutic molecule of interest and of the hydrogel with intrinsic anti-inflammatory properties.

1. A composition for oral administration for treatment of gastrointestinal disease or injury of a subject, said composition comprising:
nanoemulsion particles (NEs) comprising a hydrophobic liquid core and at least one hydrophobic surfactant (S1) and at least one hydrophilic surfactant (S2) in the external layer, said surfactants being nonionic polar surfactants surrounding the core; wherein the NEs are combined with a self-assembling peptide.

2. The composition of claim 1, wherein the self-assembling peptide, combined with NEs, is in a dry form, and which when being hydrated at a slightly acidic or near-neutral pH assembles into a hydrogel matrix enveloping the NEs, thereby adhering to and/or delivering the active agent to the intestinal walls upon oral administration to the subject.

3. The composition of claim 1, wherein the core comprises a therapeutically active agent.

4. The composition of claim 1, wherein the composition is administered in the form of capsule or tablet.

5. The composition of claim 4, wherein the capsule or the tablet slowly dissolves in the stomach of the subject or in post-stomach intestine, thereby forming a depot in the gastrointestinal tract of the subject, thereby releasing the composition into the intestine of the subject.

6. The composition of claim 1, wherein the surfactant comprises S1 being Polyoxyethylene(40) stearate (Myrj®52) and S2 being oleoyl polyoxyl-6 glycerides (Labrafil®1944CS).

7. The composition of claim 1, wherein the ratio of S1 to S2 is between 1 and 5 wt/wt.

8. The composition of claim 1, where the self-assembling peptide is any one of the peptides chosen from Table 1 (SEQ ID NOs: 1-24).

9. The composition of claim 8, wherein the self-assembling peptide is provided in a dry form.

10. The composition of claim 8, wherein the self-assembling peptide is RADA16 (SEQ ID NO:1).

11. The composition of claim 8, wherein the self-assembling peptide is IEIK13 (SEQ ID NO:2).

12. The composition of claim 1, wherein the active agent is chosen from the group consisting of Tofacitinib, Curcumin, and Budesonide

13. A method of treating a gastrointestinal disease or injury by orally administering to a subject in need thereof the composition of claim 1, thereby treating a gastrointestinal disease or injury.

14. The method of claim 13, wherein the disease being treated is Inflammatory Bowel Disease, Crohn's Disease, Ulcerative Colitis, or lesions resulting from surgery or cancer treatment.

15. A method of making the composition of claim 1, the method comprising:
a) creating nanoemulsion particles (NEs) comprising a hydrophobic liquid core and at least one hydrophobic surfactant (S1) and at least hydrophilic surfactant (S2) in the external layer, said surfactants being nonionic polar surfactants surrounding the core, said core comprising a therapeutically active agent, thereby producing the first resulting composition; and
b) combining the first resulting composition with one or more a self-assembling peptides present in solution, said peptide, when being hydrated at near-neutral pH, capable of assembling into a hydrogel, thereby producing a composite material.

16. The method of claim 15, wherein the composite material is dried to create a powder or a “cake”.

17. The method of claim 15, further comprising formulating the material suitable for oral administration to a subject.

18. The method of claim 15, wherein the composite material is encapsulated in a capsule or formulated as a tablet, either one capable of providing a depot for the composite material release, when said capsule or tablet is disposed in a subject's gastrointestinal tract.

19. The composition of claim 15, wherein the core comprises a therapeutically active agent.

20. The composition of claim 1, wherein the active agent is chosen from the group consisting of S1P receptor modulators, other JAK inhibitors, CCR9 antagonists, alpha-4 integrin antagonists, and immunomodulators.
